(19) United States
(12) Patent Application Publication
(10) Pub. No.: US 2026/0191232 A1
(43) Pub. Date: Jul. 9, 2026

(54) CO-GRANULATE FOR ANIMAL FEED

(71) Applicant: NOVOZYMES A/S, Bagsvaerd (DK)

(72) Inventors: Flemming BORUP, Bagsvaerd (DK); Ivan GAYTAN PEREZ, Kaiseraugst (CH)

(73) Assignee: NOVOZYMES A/S, Bagsvaerd (DK)

(21) Appl. No.: 19/132,685

(22) PCT Filed: Dec. 7, 2023
(86) PCT No.: PCT/EP2023/084745

§ 371 Date: May 23, 2025

(30) Foreign Application Priority Data
Dec. 8, 2022 (WO) PCT/EP2022/085064
Sep. 29, 2023 (EP) 23200769.0

(51) Int. Cl. A23K 20/189; A23K 40/30; C12N 9/16; C12N 9/24; C12N 9/26; C12N 9/42; C12N 9/50; C12N 9/98

(52) U.S. Cl. CPC A23K 20/189; A23K 40/30; C12N 9/16; C12N 9/2411; C12N 9/244; C12N 9/248; C12N 9/2482; C12N 9/2488; C12N 9/50; C12N 9/98; C12Y 302/01006; C12Y 302/01008

(57) ABSTRACT

An animal feed comprising an animal feed additive, said additive comprising co-granules, each co-granule comprising at least two classes of enzymes, said co-granules comprising a core unit and a protective coating such that the dose of each of the enzymes in the feed is set in each granule rather than by the level of homogeneity of single-enzyme granules or by the efficiency of the mixing.

### REFERENCE TO SEQUENCE LISTING

This application contains a Sequence Listing in computer readable form. The computer readable form is incorporated herein by reference.

### FIELD OF THE INVENTION

The present invention relates to animal feed accurately dosed using co-granules comprising two or more enzymes.

### BACKGROUND OF THE INVENTION

Typically, when an animal feed has raw materials whereby two or more enzyme activities are needed to optimize their nutritional contribution, two (or more) individual enzyme products are mixed into the premix or feed. When two enzyme products are to be mixed in a premix or into animal feed, there is a difficulty in dosing the enzymes properly. When two granules, each comprising an enzyme, are mixed, mixing them adequately for the proper dosing is problematic. Furthermore, differences in the shape, size and other physical features may impact their distribution within a mixture of co-granules. It is therefore difficult for proper dosing of the animals into a premix or into an animal feed.

There is a need is in the art to be able to dose combinations of enzymes in a facile manner, with reliable dosing of each enzyme in both relative and absolute amounts.

### SUMMARY OF THE INVENTION

A first aspect of the invention is directed to a co-granule comprising a core and a coating, wherein the core comprises at least two enzyme classes. A further aspect is directed to co-granules comprising at least two classes of enzymes, said co-granules comprising a core unit and a coating, wherein the co-granule core comprises the at least two classes of enzymes, wherein the co-granule is homogenous such that i) the quantity of each enzyme in one co-granule is 80-120% of the quantity of the same enzyme in another co-granules; and/or ii) wherein the ratio of the at least two classes of enzymes within a co-granule is 80% to 120% the ratio of the same two enzymes in the other co-granule.

A further aspect of the invention is directed to a granulate composition comprising co-granules, the co-granule comprising at least two classes of enzymes, said co-granules comprising a core unit and a coating, wherein the co-granule core comprises the at least two classes of enzymes, wherein the granulate composition has a homogeneity such that i) the quantity of each enzyme in one co-granule is 80-120% of the quantity of the same enzyme in at least 90% of the co-granules in said composition; and/or ii) wherein the ratio of the at least two classes of enzymes within a co-granule is 80% to 120% the ratio of the same two enzymes within at least 90% of the co-granules in said composition.

An aspect of the invention is directed to a granulate composition comprising co-granules, the co-granule comprising at least two classes of enzymes, said co-granules comprising a core unit and a coating, wherein the co-granule core comprises the at least two classes of enzymes, wherein the at least two classes of enzymes are selected from the group consisting of a phytase having at least 80% sequence identity, such as at least 85%, at least 90%, at least 95%, such as at least 99% or 100% sequence identity to SEQ ID NO: 18 and a xylanase having at least 80% sequence identity, such as at least 85%, at least 90%, at least 95%, such as at least 99% or 100% sequence identity to SEQ ID NO:1.

An aspect of the invention is directed to a granulate composition comprising co-granules, the co-granule comprising at least two classes of enzymes, said co-granules comprising a core unit and a coating, wherein the co-granule core comprises the at least two classes of enzymes, wherein the at least two classes of enzymes are selected from the group consisting of an amylase having at least 80% sequence identity, such as at least 85%, at least 90%, at least 95%, such as at least 99% or 100% sequence identity to SEQ ID NO:2 and a protease having at least 80% sequence identity, such as at least 85%, at least 90%, at least 95%, such as at least 99% or 100% sequence identity to SEQ ID NO:16.

An aspect of the invention is directed to a granulate composition comprising co-granules, the co-granule comprising at least two classes of enzymes, said co-granules comprising a core unit and a coating, wherein the co-granule core comprises the at least two classes of enzymes, wherein the at least two classes of enzymes are selected from the group consisting of an endo 1,4-beta-xylanase having at least 80% sequence identity, such as at least 85%, at least 90%, at least 95%, such as at least 99% or 100% sequence identity to SEQ ID NO:3, optionally with an endo-1,3(4)-beta-glucanase having at least 80% sequence identity, such as at least 85%, at least 90%, at least 95%, such as at least 99% or 100% sequence identity to SEQ ID NO:4 and an endo 1,4-beta-glucanase having at least 80% sequence identity, such as at least 85%, at least 90%, at least 95%, such as at least 99% or 100% sequence identity to SEQ ID NO:4; and further comprising a protease having at least 80% sequence identity, such as at least 85%, at least 90%, at least 95%, such as at least 99% or 100% sequence identity to SEQ ID NO: 15.

An aspect of the invention is directed to a granulate composition comprising co-granules, the co-granule comprising at least two classes of enzymes, said co-granules comprising a core unit and a coating, wherein the co-granule core comprises the at least two classes of enzymes, wherein the at least two classes of enzymes are selected from the group consisting of an endo 1,4-beta-xylanase having at least 80% sequence identity, such as at least 85%, at least 90%, at least 95%, such as at least 99% or 100% sequence identity to SEQ ID NO: 3, an endo-1,3(4)-beta-glucanase and endo 1,4-beta-glucanase and a phytase having at least 80% sequence identity, such as at least 85%, at least 90%, at least 95%, such as at least 99% or 100% sequence identity to SEQ ID NO:18.

An aspect of the invention is directed to a granulate composition comprising co-granules, the co-granule comprising at least two classes of enzymes, said co-granules comprising a core unit and a coating, wherein the co-granule core comprises the at least two classes of enzymes, wherein the at least two classes of enzymes are selected from the group consisting of an endo 1,4-beta xylanase having at least 80% sequence identity, such as at least 85%, at least 90%, at least 95%, such as at least 99% or 100% sequence identity to SEQ ID NO:3, an endo 1,4-beta xylanase xylanase having at least 80% sequence identity, such as at least 85%, at least 90%, at least 95%, such as at least 99% or 100% sequence identity to SEQ ID NO:4; an endo 1,3(4) beta-glucanase having at least 80% sequence identity, such as at least 85%, at least 90%, at least 95%, such as at least 99% or 100% sequence identity to SEQ ID NO:5; and a phytase having at least 80% sequence identity, such as at least 85%, at least 90%, at least 95%, such as at least 99% or 100% sequence identity to SEQ ID NO:19.

An aspect of the invention is directed to a granulate composition comprising co-granules, the co-granule comprising at least two classes of enzymes, said co-granules comprising a core unit and a coating, wherein the co-granule core comprises the at least two classes of enzymes, wherein the at least two classes of enzymes are selected from the group consisting of a xylanase having at least 80% sequence identity, such as at least 85%, at least 90%, at least 95%, such as at least 99% or 100% sequence identity to SEQ ID NO:1 and a phytase of a phytase having at least 80% sequence identity, such as at least 85%, at least 90%, at least 95%, such as at least 99% or 100% sequence identity to SEQ ID NO:19.

An aspect of the invention is directed to a granulate composition comprising co-granules, the co-granule comprising at least two classes of enzymes, said co-granules comprising a core unit and a coating, wherein the co-granule core comprises the at least two classes of enzymes, wherein the at least two classes of enzymes are selected from the group consisting of an endo 1,4-beta-xylanase, such as an endo 1,4-beta-xylanase having at least 80% sequence identity, such as at least 85%, at least 90%, at least 95%, such as at least 99% or 100% sequence identity to SEQ ID NO:3; an endo-1,3(4)-beta-glucanase, such as an endo-1,3(4)-beta-glucanase having at least 80% sequence identity, such as at least 85%, at least 90%, at least 95%, such as at least 99% or 100% sequence identity to SEQ ID NO:4, an endo 1,4-beta-glucanase, an xylanase, such as a xylanase having at least 80% sequence identity, such as at least 85%, at least 90%, at least 95%, such as at least 99% or 100% sequence identity to SEQ ID NO:1, and optionally a beta-glucanase, and/or an amylase, such as an amylase having at least 80% sequence identity, such as at least 85%, at least 90%, at least 95%, such as at least 99% or 100% sequence identity to SEQ ID NO:2.

An aspect of the invention is directed to a granulate composition comprising co-granules, the co-granule comprising at least two classes of enzymes, said co-granules comprising a core unit and a coating, wherein the co-granule core comprises the at least two classes of enzymes, wherein the at least two classes of enzymes are selected from the group consisting of a beta-glucanase, such as a beta-glucanase having at least 80% sequence identity such as at least 85%, at least 90%, at least 95%, such as at least 99% or 100% sequence identity to SEQ ID NO:8 and an amylase, such as an amylase having at least 80% sequence identity such as at least 85%, at least 90%, at least 95%, such as at least 99% or 100% sequence identity to SEQ ID NO:2; and a protease, such as a protease having at least 80% sequence identity such as at least 85%, at least 90%, at least 95%, such as at least 99% or 100% sequence identity to SEQ ID NO:15 or SEQ ID NO:16.

Another aspect of the invention is directed to animal feed comprising an animal feed additive, said additive comprising co-granules, each co-granule comprising at least two classes of enzymes, said co-granules comprising a core unit and a protective coating.

Another aspect of the invention is directed to a method of manufacturing a co-granule comprising combining two enzyme classes onto a core and coating the enzyme-containing core with a coating.

### BRIEF DESCRIPTION OF DRAWINGS

FIG. 1 illustrates a traditional way of combining two enzymes wherein the single enzyme granules are mixed together. The degree of homogeneity will depend on a number of factors including the relative amount of each granule, the granule size, shape and coating, the differences in settling of the granules during storage or transportation and the efficiency of the mixing of the granules.

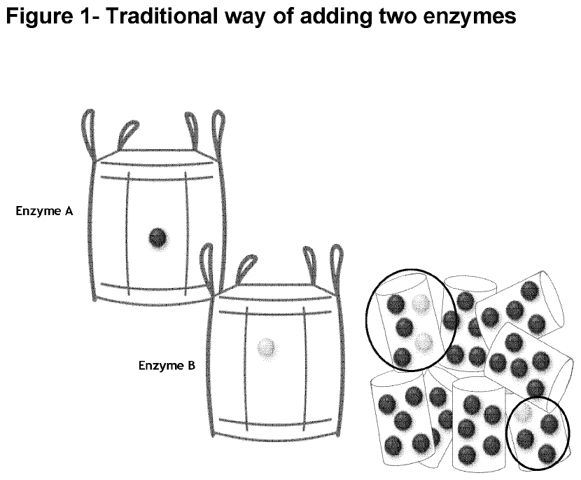

FIG. 2 illustrates a co-granule with a multiple enzyme-containing core and a coating.

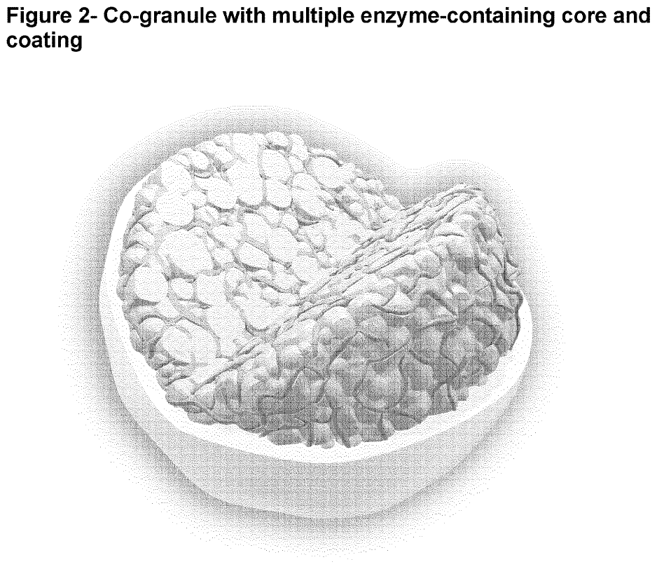

FIG. 3 illustrates co-granules providing homogeneity in the distribution of the enzymes in a granulate composition for use in a pre-mix or feed composition. The dose of each enzyme is controlled by granulation method wherein the two or more enzyme classes are coated on the core of the granule thereby ensuring that the relative and absolute doses of the enzymes are as desired.

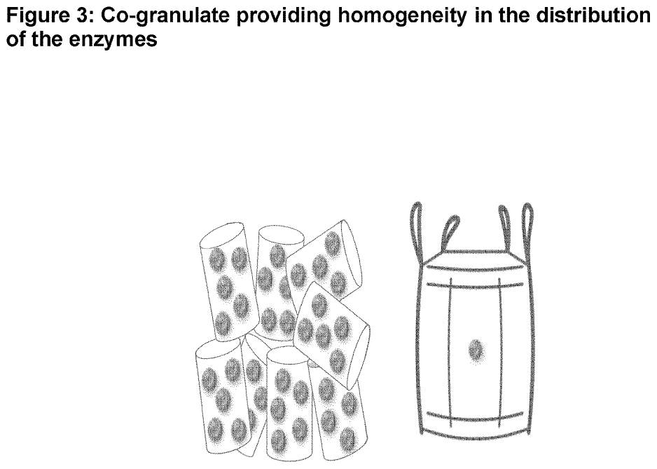

FIG. 4 illustrates the contrast of the distribution of enzymes in granules compared to in co-granules.

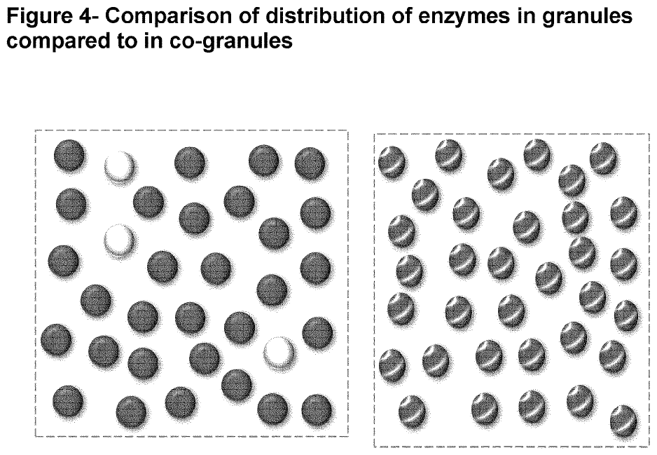

### BRIEF DESCRIPTION OF THE SEQUENCES

SEQ ID NO:1 is an endo-1,4-beta-xylanase originating from Thermomyces lanuginosus.

SEQ ID NO:2 is an amylase originating from Bacillus amyloliquefaciens.

SEQ ID NO:3 is a GH11-5 endo-b-1,4-xylanase originating from Trichoderma reesei.

SEQ ID NO:4 is an endoglucanase I originating from Trichoderma reesei.

SEQ ID NO:5 is a carbohydrase originating from Trichoderma reesei.

SEQ ID NO:6 is a carbohydrase originating from Trichoderma reesei.

SEQ ID NO:7 is a beta-xylosidase originating from Trichoderma reesei.

SEQ ID NO:8 is an endo-1,3(4)-beta-glucanase originating from Aspergillus aculeatus.

SEQ ID NO:9 is a polygalacturonase originating from Aspergillus aculeatus.

SEQ ID NO:10 is a beta-glucanase originating from Aspergillus aculeatus.

SEQ ID NO: 11 is a carbohydrase originating from Aspergillus aculeatus.

SEQ ID NO:12 is a carbohydrase originating from Aspergillus aculeatus.

SEQ ID NO:13 is a carbohydrase originating from Aspergillus aculeatus.

SEQ ID NO:14 is a carbohydrase originating from Aspergillus aculeatus.

SEQ ID NO:15 is a protease originating from Nocardiopsis prasina.

SEQ ID NO:16 is a protease originating from Bacillus sp-11238.

SEQ ID NO:17 is an mannanase originating from Talaromyces Leycettanus.

SEQ ID NO:18 is a phytase originating from Citrobacter braakii.

SEQ ID NO:19 is a phytase originating from Citrobacter braakii.

SEQ ID NO:20 is an muramidase originating from Acremonium alkalophilum.

SEQ ID NO:21 is an endo-1,4-beta-xylanase originating from Bacillus subtilis.

SEQ ID NO:22 is a xylanase originating from Trichoderma reesei.

SEQ ID NO:23 is a xylanase originating from Thermopolyspora flexuosa.

SEQ ID NO:24 is a beta-mannanase originating from Paenibacillus woosongensis.

SEQ ID NO:25 is an endoxylanase originating from Trichoderma reesei.

SEQ ID NO:26 is an endo-1,4-beta-xylanase originating from Trichoderma viride.

SEQ ID NO:27 is a glycoside hydrolase in family 7/1, 4-beta cellobiohydrolase originating from Talaromyces funiculosus.

SEQ ID NO:28 is a 1,4-beta cellobiohydrolase originating from Talaromyces funiculosus.

SEQ ID NO:29 is a glycoside hydrolase family 11/12 originating from Talaromyces funiculosus.

SEQ ID NO:30 is a carbohydrase originating from Talaromyces funiculosus.

SEQ ID NO:31 is an arabinofuranosidase GH62-1 originating from Talaromyces funiculosus.

SEQ ID NO:32 is a protease/subtilisin originating from Bacillus amyloliquefaciens.

SEQ ID NO:33 is an endo-1,4-beta-xylanase (Xyn1) originating from Rasamsonia emersonii.

SEQ ID NO:34 is an alpha amylase originating from Aspergillus niger.

SEQ ID NO:35 is an endo-1,4-beta-xylanase GH11A originating from Trichoderma reesei.

SEQ ID NO:36 is an alpha-amylase originating from Bacillus licheniformis.

SEQ ID NO:37 is a subtilisin originating from Bacillus amyloliquefaciens.

SEQ ID NO:38 is an endo-1,4-beta-xylanase GH11A originating from Trichoderma reesei.

SEQ ID NO:39 is a protease originating from Streptomyces fradiae.

SEQ ID NO:40 is a peptidase S8/S53/serine proteases originating from Bacillus licheniformis.

SEQ ID NO:41 is a thermostabilized PE variant of phytase originating from Buttiauxella sp.

SEQ ID NO:42 is a thermostabilized PE variant of phytase originating from Buttiauxella sp.

SEQ ID NO:43 is a phytase originating from Hafnia.

SEQ ID NO:44 is a wild-type AppA2 phytase originating from Escherichia coli.

SEQ ID NO:45 is a variant of SEQ ID NO: 44.

SEQ ID NO:46 is a phytase originating from Escherichia coli.

SEQ ID NO:47 is synthetic phytase.

SEQ ID NO:48 is a phytase originating from Escherichia coli.

SEQ ID NO: 49 is a xylanase originating from Trichoderma reesi.

SEQ ID NO:50 is an amylase originating from Bacillus licheniformis.

SEQ ID NO:51 is a protease originating from Bacillus amyloliquefaciens.

SEQ ID NO:52 is a phytase originating from Buttiauxella sp.

SEQ ID NO:53 is a phytase originating from Hafnia sp. Mollaretii.

SEQ ID NO:54 is a carbohydrase originating from Rasamsonia emersonii.

SEQ ID NO:55 is a phytase derived from phytase originating from Escherichia coli.

SEQ ID NO:56 is a carbohydrase originating from Trichoderma citrinoviride.

SEQ ID NO:57 is a phytase derived from phytase originating from Escherichia coli.

SEQ ID NO:58 is a carbohydrase originating from Nonomuraea flexuosa.

SEQ ID NO:59 is a phytase derived from phytase originating from Escherichia coli.

SEQ ID NO:60 is a xylanase originating from Neocallimastix patriciarum.

SEQ ID NO:61 is a protease originating from Bacillus amyloliquefaciens.

SEQ ID NO:62 is a mannanase originating from Rhizomucor miehei.

### DETAILED DESCRIPTION OF THE INVENTION

The typical way of preparing a feed comprising two or more enzymes involves dosing two or more granules, each comprising the enzymes of interest. This creates problems for dosing giving the desired amounts of each enzyme may be such that one may be combining many of one granule with few of the other type of granule. This leads to dosing problems given the distribution of the granules may not be homogenous. Homogeneity is also a problem when dosing close to equal amounts of each of the granule. Homogeneity and/or mixing problems invariably leads to dosing inconsistencies. This leads to problems for the farmers in terms of livestock size and homogeneity. Furthermore, this lack of homogeneity is sometimes compensated by increasing the dose of the enzymes to avoid low levels being distributed. This is a costly and wasteful solution by the farmer.

The inventors have remedied this dosing issue by developing a co-granule comprising at least two enzymes, so that the dose of each of the enzymes is set in each granule rather than by the level of homogeneity of single-enzyme granules or set by the efficiency of the mixing.

The inventors have developed a co-granule of two or more enzyme classes which provides a number of advantages over conventional including more convenient handling, easier inventory handling, easier application handling and better distribution and homogeneity in animal feed, so as to get the desired or regimented amount of enzyme in every representative feed sample. The invention is directed to a granulate comprising a co-granule with two or more specific enzyme activities mixed into premix or feed. The co-granules are thermal stable, contribute to gastric stability, dust free and provide a homogenous distribution of the enzymes in a feed or pre-mix composition. One unique feature that could be obtained with co-granules of the invention, is that very low dosing of an enzyme can be reliably achieved in every feed composition.

When referring to the co-granule of the present invention the term can either refer a single co-granule or several co-granules.

The co-granule of the present invention is particularly well suited for steam pelleting and as part of a steam treated pelletized feed composition. The co-granule preferably comprises a core and a coating, wherein the core comprises two enzymes and the co-granule optionally comprises a coating comprises a salt layer and/or a hydrophobic layer.

An aspect of the invention is directed to a granulate composition comprising co-granules, each co-granule comprising at least two classes of enzymes, said co-granules comprising a core unit and a protective coating, wherein the co-granule core comprises the at least classes of enzymes, wherein the granulate composition has a homogeneity such that i) the quantity of each enzyme in one co-granule is 80-120% of the quantity of the same enzyme in at least 90% of the co-granules in said composition; and/or ii) wherein the ratio of the at least two classes of enzymes within a co-granule is 80% to 120% the ratio of the same two enzymes within at least 90% of the co-granules in said composition. In a preferred embodiment, the at least two classes of enzymes are selected from the group consisting of a xylanase, an amylase, a mannanase, a carbohydrase, a cellulase, a hemi-cellulase, a polygalacturonase, a beta-glucanase, a phytase, a protease, a pectinase, and a muramidase, preferably wherein at least one of the enzymes is selected from xylanase, an amylase, a mannanase, a carbohydrase, a cellulase, a polygalacturonase and a beta-glucanase.

The at least two classes of enzymes are preferably selected from the group consisting of a xylanase, an amylase, a carbohydrase, a mannanase, a cellulase, a hemi-cellulase, a polygalacturonase, a beta-glucanase, a phytase, a protease, a pectinase and a muramidase, preferably wherein at least one of the enzymes is selected from xylanase, an amylase, a mannanase, a carbohydrase, a polygalacturonase and a beta-glucanase. In a suitable embodiment, the at least two classes of enzymes are preferably selected from the group consisting of a carbohydrase, a phytase, a muramidase, and a protease, most preferably where at least one of the enzymes is a carbohydrase.

The co-granules typically comprise a combination selected from the group consisting of:
a. a combination of a phytase and a xylanase;
b. a combination of an amylase and a protease;
c. a combination of a xylanase and a protease;
d. a combination of a phytase, a protease and a xylanase;
e. a combination of an endo 1,4-beta-xylanase, an endo-1,3(4)-beta-glucanase, a endo-1,4-beta-glucanase, a beta-glucanase and a protease;
f. a combination of a 1,4-beta-xylanase, an endo-1,4-beta-glucanase and a phytase;
g. a combination of a 1,4-beta-xylanase, an endo-1,3(4)-beta-glucanase, an endo 1,4-beta-glucanase and a phytase;
h. a combination of an endo 1,4-beta-xylanase and an amylase;
i. a combination of an endo 1,4-beta-xylanase, a endo 1,4-beta-glucanase and an amylase;
j. a combination of an endo 1,4-beta-xylanase, endo-1,3(4)-beta-glucanase and endo 1,4-beta-glucanase with the xylanase beta-glucanase (200 ppm) and with the amylase; and
k. a combination of a beta-glucanase, an amylase and a protease.
l. a combination of a xylanase, an amylase and a protease;
m. a combination of a xylanase, an amylase, a protease and a phytase;
n. a combination of a protease and a phytase;
o. a combination of a phytase and a carbohydrase;
p. a combination of a phytase and a mannanase;
q. a combination of a mannanase and a carbohydrase; and
r. a combination of a phytase, xylanase, protease and mannanase.

The co-granules preferably comprise a combination selected from the group consisting of:
a. a combination of a phytase and a xylanase;
b. a combination of an amylase and a protease;
c. a combination of a xylanase and a protease;
d. a combination of a xylanase, a glucanase and a protease;
e. a combination of a xylanase and a glucanase;
f. a combination of a xylanase, a glucanase and a phytase;
g. a combination of a xylanase and an amylase;
h. a combination of a xylanase, a glucanase and an amylase;
i. a combination of a phytase and a protease;
j. a combination of a phytase and an amylase; and
k. a combination of an amylase and a glucanase.

The co-granules typically comprise a combination selected from the group consisting of:
a. a combination of at least one carbohydrase and a protease
b. a combination of at least one carbohydrase and a phytase
c. a combination of a phytase and a protease
d. a combination of at least two classes of carbohydrases, preferably originating from different sources.

The co-granules typically comprise a combination of two or more enzymes selected from the group consisting of a polypeptide having at least 80% sequence identity such as at least 90% sequence identity, such as at least 95% sequence identity, such as at least 96%, such as at least 97%, such as at least 98%, such as at least 99%, such as 100% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO:34, SEQ ID NO: 35, SEQ ID NO: 35, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61 and SEQ ID NO: 62. Preferably, at least one enzyme is a carbohydrase or a phytase.

Preferably, in embodiments where all of the enzymes in the co-granule are carbohydrases, at least two carbohydrases are from different sources.

The granulate composition may comprise a co-granule comprising a protease. Preferably, the protease is selected from the group consisting of a polypeptide having at least 80% sequence identity such as at least 90% sequence identity, such as at least 95% sequence identity, such as at least 96%, such as at least 97%, such as at least 98%, such as at least 99%, such as 100% sequence identity to SEQ ID NO: 15, SEQ ID NO: 16, SEQ ID NO: 32, SEQ ID NO: 39, SEQ ID NO: 40, SEQ ID NO: 51 and SEQ ID NO:61.

In a preferred embodiment of the invention, the protease is the protease from a commercially available protease product used in animal feed, more preferably the protease is from any one of Ronozyme® PROACT, Ronozyme® PROACT360, Cibenza®, PoultryGro® and Axtra® Pro. As known to the person skilled in the art, polypeptide sequences often comprise signal sequences, N-terminal and/or C-terminal sequences for purposes of expression and manipulation. Accordingly, the polypeptide sequences of the commercially available products may be varied at either terminal within the scope of the present invention. In one embodiment, the co-granule comprises a carbohydrase and a protease selected from the group consisting of a polypeptide having at least 80% sequence identity such as at least 90% sequence identity, such as at least 95% sequence identity, such as at least 96%, such as at least 97%, such as at least 98%, such as at least 99%, such as 100% sequence identity to SEQ ID NO: 15, SEQ ID NO: 16, SEQ ID NO: 32, SEQ ID NO: 39, SEQ ID NO: 40, SEQ ID NO: 51 and SEQ ID NO:61.

The granulate composition may comprise a co-granule comprising an amylase. Preferably, the amylase is selected from the group consisting of a polypeptide having at least 80% sequence identity such as at least 90% sequence identity, such as at least 95% sequence identity, such as at least 96%, such as at least 97%, such as at least 98%, such as at least 99%, such as 100% sequence identity to SEQ ID NO: 2, SEQ ID NO: 34, SEQ ID NO:36 and SEQ ID NO:50.

In a preferred embodiment of the invention, the amylase is the amylase from a commercially available amylase product used in animal feed, more preferably the amylase is from any one of Ronozyme® HiStarch, Axtra® XB and Natugrain®. As known to the person skilled in the art, polypeptide sequences often comprise signal sequences, N-terminal and/or C-terminal sequences for purposes of expression and manipulation. Accordingly, the polypeptide sequences of the commercially available products may be varied at either terminal within the scope of the present invention.

The granulate composition may comprise a co-granule comprising a xylanase. Preferably, the xylanase is selected from the group consisting of a polypeptide having at least 80% sequence identity such as at least 90% sequence identity, such as at least 95% sequence identity, such as at least 96%, such as at least 97%, such as at least 98%, such as at least 99%, such as 100% sequence identity to SEQ ID NO:1, SEQ ID NO:3, SEQ ID NO:21, SEQ ID NO:22, SEQ ID NO:23, SEQ ID NO:25, SEQ ID NO:26, SEQ ID NO:33, SEQ ID NO:35, SEQ ID NO:38, SEQ ID NO: 49 and SEQ ID NO:60.

In a preferred embodiment of the invention, the xylanase is the xylanase from a commercially available xylanase product used in animal feed, more preferably the xylanase is from any one of Ronozyme® WX, Ronozyme® Multigrain, Belfeed® B1100, Danisco® Xylanase, Econase® XT, Hostazyme® X, Axtra® XB, Axtra® XAP and Natugrain®. As known to the person skilled in the art, polypeptide sequences often comprise signal sequences, N-terminal and/or C-terminal sequences for purposes of expression and manipulation. Accordingly, the polypeptide sequences of the commercially available products may be varied at either terminal within the scope of the present invention.

The granulate composition may comprise a co-granule comprising a phytase. Preferably, the phytase is selected from the group consisting of a polypeptide having at least 80% sequence identity such as at least 90% sequence identity, such as at least 95% sequence identity, such as at least 96%, such as at least 97%, such as at least 98%, such as at least 99%, such as 100% sequence identity to SEQ ID NO:18, SEQ ID NO:19, SEQ ID NO:41, SEQ ID NO:42, SEQ ID NO:43, SEQ ID NO:44, SEQ ID NO:45, SEQ ID NO:48, SEQ ID NO: 52, SEQ ID NO: 53, SEQ ID NO: 55, SEQ ID NO: 57, and SEQ ID NO: 58. In a preferred embodiment, the phytase is the phytase from a commercially available product used in animal feed, such as Ronozyme® HiPhos, Ronozyme® HiPhorius, OptiPhos®, OptiPhos® Plus, Apsa® Phytafeed®, Naturphos® E, Phyzyme®, Quantum®, Quantum® Blue, and Axtra® PHY.

The granulate composition may comprise a co-granule comprising a carbohydrase. Preferably, the carbohydrase is selected from the group consisting of a polypeptide having at least 80% sequence identity such as at least 90% sequence identity, such as at least 95% sequence identity, such as at least 96%, such as at least 97%, such as at least 98%, such as at least 99%, such as 100% sequence identity to SEQ ID NO:5, SEQ ID NO:6, SEQ ID NO:11, SEQ ID NO:13, SEQ ID NO:14, SEQ ID NO:30, SEQ ID NO: 54, SEQ ID NO:56 and SEQ ID NO:58.

In a preferred embodiment, the co-granule comprises at least one, preferably two or more enzymes selected from the group consisting of:
a. a phytase selected from the group consisting of a polypeptide having at least 90% sequence identity, such as at least 95%, such as at least 98%, such as at least 99%, such as 100% sequence identity to SEQ ID NO:18, SEQ ID NO:19, SEQ ID NO:41, SEQ ID NO:42, SEQ ID NO:43, SEQ ID NO:44, SEQ ID NO:45, SEQ ID NO:48, SEQ ID NO: 52, SEQ ID NO: 53, SEQ ID NO: 55, SEQ ID NO: 57, and SEQ ID NO: 58;
b. a xylanase selected from the group consisting of a polypeptide having at least 90% sequence identity, such as at least 95%, such as at least 98%, such as at least 99%, such as 100% sequence identity to SEQ ID NO:1, SEQ ID NO:3, SEQ ID NO:21, SEQ ID NO:22, SEQ ID NO:23, SEQ ID NO:25, SEQ ID NO:26, SEQ ID NO:33, SEQ ID NO:35, SEQ ID NO:38, SEQ ID NO: 49 and SEQ ID NO:60;
c. a protease selected from the group consisting of SEQ ID NO: 15, SEQ ID NO: 16, SEQ ID NO: 32, SEQ ID NO: 37, SEQ ID NO: 39, SEQ ID NO: 40, SEQ ID NO: 51 and SEQ ID NO: 61;
d. an amylase selected from the group consisting of SEQ ID NO: 2, SEQ ID NO: 36 and SEQ ID NO:50;
e. a carbohydrase selected from the group consisting of SEQ ID NO: 5, SEQ ID NO: 6, SEQ ID NO: 11, SEQ ID NO: 12, SEQ ID NO: 13, SEQ ID NO: 14, SEQ ID NO: 30, SEQ ID NO: 54, SEQ ID NO: 56 and SEQ ID NO: 58; and
f. a mannanase selected from the group consisting of SEQ ID NO: 17, SEQ ID NO: 24 and SEQ ID NO:62.

In one embodiment, the co-granule comprises a carbohydrase and a phytase selected from the group consisting of a polypeptide having at least 80% sequence identity such as at least 90% sequence identity, such as at least 95% sequence identity, such as at least 96%, such as at least 97%, such as at least 98%, such as at least 99%, such as 100% sequence identity to SEQ ID NO:5, SEQ ID NO:6, SEQ ID NO:11, SEQ ID NO:13, SEQ ID NO:14, SEQ ID NO:30, SEQ ID NO: 54, SEQ ID NO:56, SEQ ID NO:58, SEQ ID NO:18, SEQ ID NO:19, SEQ ID NO:41, SEQ ID NO:42, SEQ ID NO:43, SEQ ID NO:44, SEQ ID NO:45 and SEQ ID NO:48. In one embodiment, the co-granule comprises at least two classes of enzymes, wherein the at least two classes of enzymes are selected from the group consisting of
i. 12000 FYT/g of a phytase having at least 80% sequence identity, such as at least 85%, at least 90%, at least 95%, such as at least 99% or 100% sequence identity to SEQ ID NO: 18 and 800 FXU-W/g of a xylanase having at least 80% sequence identity, such as at least 85%, at least 90%, at least 95%, such as at least 99% or 100% sequence identity to SEQ ID NO:1;
ii. 10000 FYT/g of a phytase having at least 80% sequence identity, such as at least 85%, at least 90%, at least 95%, such as at least 99% or 100% sequence identity to SEQ ID NO: 18 and 360 FXU-W/g of a xylanase having at least 80% sequence identity, such as at least 85%, at least 90%, at least 95%, such as at least 99% or 100% sequence identity to SEQ ID NO:1;
iii. 641 KNU—Kilo Novo Units of an amylase having at least 80% sequence identity, such as at least 85%, at least 90%, at least 95%, such as at least 99% or 100% sequence identity to SEQ ID NO:2 and 240.000 NFP of a protease having at least 80% sequence identity, such as at least 85%, at least 90%, at least 95%, such as at least 99% or 100% sequence identity to SEQ ID NO:16;
iv. 1.080 units of an endo 1,4-beta-xylanase having at least 80% sequence identity, such as at least 85%, at least 90%, at least 95%, such as at least 99% or 100% sequence identity to SEQ ID NO:3, optionally with 320 units of an endo-1,3(4)-beta-glucanase having at least 80% sequence identity, such as at least 85%, at least 90%, at least 95%, such as at least 99% or 100% sequence identity to SEQ ID NO:4 and 320 units of an endo 1,4-beta-glucanase having at least 80% sequence identity, such as at least 85%, at least 90%, at least 95%, such as at least 99% or 100% sequence identity to SEQ ID NO:4; and further comprising 60.000 PROT-Keratinase Units of a protease having at least 80% sequence identity, such as at least 85%, at least 90%, at least 95%, such as at least 99% or 100% sequence identity to SEQ ID NO: 15;
v. a combination of 900 units of an endo 1,4-beta-xylanase having at least 80% sequence identity, such as at least 85%, at least 90%, at least 95%, such as at least 99% or 100% sequence identity to SEQ ID NO: 3, an endo-1,3(4)-beta-glucanase and endo 1,4-beta-glucanase and 10000 FYT/g of a phytase having at least 80% sequence identity, such as at least 85%, at least 90%, at least 95%, such as at least 99% or 100% sequence identity to SEQ ID NO:18;
vi. 900 units of an endo 1,4-beta xylanase having at least 80% sequence identity, such as at least 85%, at least 90%, at least 95%, such as at least 99% or 100% sequence identity to SEQ ID NO:3, an endo 1,4-beta xylanase xylanase having at least 80% sequence identity, such as at least 85%, at least 90%, at least 95%, such as at least 99% or 100% sequence identity to SEQ ID NO:4; an endo 1,3(4) beta-glucanase having at least 80% sequence identity, such as at least 85%, at least 90%, at least 95%, such as at least 99% or 100% sequence identity to SEQ ID NO:5; and 7500 FYT/g of a phytase having at least 80% sequence identity, such as at least 85%, at least 90%, at least 95%, such as at least 99% or 100% sequence identity to SEQ ID NO:19;
vii. 500 FXU-W/g of a xylanase having at least 80% sequence identity, such as at least 85%, at least 90%, at least 95%, such as at least 99% or 100% sequence identity to SEQ ID NO:1 and 7500 FYT/g of a phytase of a phytase having at least 80% sequence identity, such as at least 85%, at least 90%, at least 95%, such as at least 99% or 100% sequence identity to SEQ ID NO:19;
viii. an endo 1,4-beta-xylanase, such as an endo 1,4-beta-xylanase having at least 80% sequence identity, such as at least 85%, at least 90%, at least 95%, such as at least 99% or 100% sequence identity to SEQ ID NO:3; an endo-1,3(4)-beta-glucanase, such as an endo-1,3(4)-beta-glucanase having at least 80% sequence identity, such as at least 85%, at least 90%, at least 95%, such as at least 99% or 100% sequence identity to SEQ ID NO:4, an endo 1,4-beta-glucanase, an xylanase, such as a xylanase having at least 80% sequence identity, such as at least 85%, at least 90%, at least 95%, such as at least 99% or 100% sequence identity to SEQ ID NO:1, and optionally a beta-glucanase, and/or an amylase, such as an amylase having at least 80% sequence identity, such as at least 85%, at least 90%, at least 95%, such as at least 99% or 100% sequence identity to SEQ ID NO:2; and
ix. a beta-glucanase, such as a beta-glucanase having at least 80% sequence identity such as at least 85%, at least 90%, at least 95%, such as at least 99% or 100% sequence identity to SEQ ID NO:8 and an amylase, such as an amylase having at least 80% sequence identity such as at least 85%, at least 90%, at least 95%, such as at least 99% or 100% sequence identity to SEQ ID NO:2; and a protease, such as a protease having at least 80% sequence identity such as at least 85%, at least 90%, at least 95%, such as at least 99% or 100% sequence identity to SEQ ID NO:15 or SEQ ID NO:16

As stated, in one embodiment, the granulate composition comprises a co-granulate comprising a xylanase, an amylase and a protease. In a suitable embodiment thereof, the xylanase is a polypeptide selected from the group consisting of SEQ ID NO: 49 (it is available from DuPont®); the amylase is a polypeptide selected from the group consisting of SEQ ID NO:50 it is available from DuPont; and the protease is a polypeptide selected from the group consisting of SEQ ID NO:51 (it is available from DuPont® under the tradename AxtraPro®). As stated, in one embodiment, the granulate composition comprises a co-granulate comprising a xylanase, an amylase, a protease and a phytase. In a suitable embodiment thereof, the xylanase is a polypeptide selected from the group consisting of SEQ ID NO: 49 (it is available from DuPont®); the amylase is a polypeptide selected from the group consisting of SEQ ID NO:50 it is available from DuPont®; the protease is a polypeptide selected from the group consisting of SEQ ID NO:51 (it is available from DuPont® under the tradename AxtraPro); and the phytase is a polypeptide selected from the group consisting of SEQ ID NO:52 (it is available from Dupont® under the tradename Axtra® Phy Gold). As stated, in one embodiment, the granulate composition comprises a co-granulate comprising a xylanase and a phytase. In a suitable embodiment thereof, the xylanase is a polypeptide selected from the group consisting of SEQ ID NO: 49 (it is available from DuPont); and the phytase is a polypeptide selected from the group consisting of SEQ ID NO:52 (it is available from Dupont® under the tradename Axtra® Phy Gold).

As stated, in one embodiment, the granulate composition comprises a co-granulate comprising a phytase and a carbohydrase. In a suitable embodiment thereof, the phytase is a polypeptide selected from the group consisting of SEQ ID NO:53 (it is available from BASF® under the tradename NatuPhos® E); and the carbohydrase is a polypeptide selected from SEQ ID NO: 54 ((it is available from BASF under the tradename Natugrain® TS). In a further suitable embodiment thereof, the phytase is a polypeptide originating from Buttiauxella sp and available from Adisseo® under the tradename Rovabio® PhyPlus or Rovabio® Advance PHY; and the carbohydrase is a polypeptide originating from Penicillium funiculosum available from Adisseo® under the tradename Rovabio® Excel or Rovabio® Advance. In a further suitable embodiment thereof, the phytase is a polypeptide selected from the group consisting of SEQ ID NO:57 and the carbohydrase is a polypeptide selected from the group consisting of SEQ ID NO:58. In an embodiment, the granulate composition comprises a co-granulate comprising a phytase and a mannanase. In a suitable embodiment thereof, the phytase is a polypeptide selected from the group consisting of SEQ ID NO:53 (it is available from BASF® under the tradename NatuPhos® E); and the mannanase is a polypeptide available from BASF® under the tradename Natupulse®. In one embodiment, the granulate composition comprises a co-granulate comprising a mannanase and a carbohydrase. In a suitable embodiment thereof, the mannanase is a polypeptide available from BASF under the tradename Natupulse® and the carbohydrase is a polypeptide selected from SEQ ID NO: 54 (it is available from BASF under the tradename Natugrain® TS).

As stated, in one embodiment, the granulate composition comprises a co-granulate comprising a phytase and a carbohydrase. In a suitable embodiment thereof, the phytase is a polypeptide selected from the group consisting of SEQ ID NO: 55 (available from Huvepharma® under the tradename Optiphos® Plus); and the carbohydrase is a polypeptide selection from SEQ ID NO:56 (available from Huvepharma® under the tradename Hostazyme®). In a suitable embodiment thereof, the co-granulate comprises two different types of carbohydrase. In a suitable embodiment thereof, the phytase is a polypeptide selected from the group consisting of SEQ ID NO: 55 (available from Huvepharma® under the tradename Optiphos® Plus); and a carbohydrase is a polypeptide selection from SEQ ID NO:56 (available from Huvepharma® under the tradename Hostazyme®) and the further carbohydrase is the carbohydrase available from Huvepharma® under the tradename Nexo®.

As stated, in one embodiment, the granulate composition comprises a co-granulate comprising a combination of a phytase, a xylanase, a protease and a mannanase. In a suitable embodiment thereof, the phytase is a polypeptide selected from the group consisting of SEQ ID NO:59 (available from VTR® Biotech under the tradename Microtech®); the xylanase is a polypeptide selected from the group consisting of SEQ ID NO:60; the protease is a polypeptide selected from the group consisting of SEQ ID NO:61; and the mannanase is a polypeptide selected from the group consisting of SEQ ID NO:62.

As stated, in one embodiment, the granulate composition comprises a co-granulate comprising a combination of a phytase, a xylanase, and a protease. In a suitable embodiment thereof, the phytase is a polypeptide selected from the group consisting of SEQ ID NO:59 (available from VTR® Biotech under the tradename Microtech®); the xylanase is a polypeptide selected from the group consisting of SEQ ID NO:60 (available from VTR® Biotech under the tradename Yiduozyme®); and the protease is a polypeptide selected from the group consisting of SEQ ID NO:61.

As stated, in one embodiment, the granulate composition comprises a co-granulate comprising a combination of a phytase and a protease. In a suitable embodiment thereof, the phytase is a polypeptide selected from the group consisting of SEQ ID NO:59 (available from VTR® Biotech under the tradename Microtech®); and the protease is a polypeptide selected from the group consisting of SEQ ID NO:61.

The granulate composition is typically such that i) the quantity of each enzyme in one co-granule is 90-110% of the quantity of the same enzyme in at least 90% of any other co-granule in said composition; and/or ii) wherein the ratio of the at least two classes of enzymes within a co-granule is 90% to 110% the ratio of the same two enzymes within at least 90% of the co-granules in said composition. The composition according to any of the preceding claims wherein i) the quantity of each enzyme in one co-granule is 80-120% of the quantity of the same enzyme in at least 95% of the co-granules in said composition; and/or ii) wherein the ratio of the at least two classes of enzymes within a co-granule is 80% to 120% the ratio of the same two enzymes within at least 95% of the co-granules in said composition. More typically, i) the quantity of each enzyme in one co-granule is 90-110% of the quantity of the same enzyme in at least 95% of the co-granules in said composition; and/or ii) wherein the ratio of the at least two classes of enzymes within a co-granule is 90% to 110% the ratio of the same two enzymes within at least 95% of the co-granules in said composition

Suitably, the co-granules comprise the at least two enzyme classes, wherein the ratio (wt/wt) of at least two enzymes is from 1:1 to 100:1. In embodiments where one of the enzymes is at a low dose, the ratio of the enzymes could be at least 5:1, such as at least 10:1, such as from 5:1 to 100:1, such as from 10:1 to 100:1.

In one embodiment, the co-granule comprises at least two classes of enzymes, wherein the at least two classes of enzymes are selected from the group consisting of
b) a wild-type phytase and xylanase;
c) an alpha amylase (EC 3.2.1.1) and an S1 protease;
d) an alpha amylase (EC 3.2.1.1) and an S8 protease;
e) a variant phytase and xylanase and further comprising enzymes suitable for insoluble and soluble arabinoxylans such as enzymes selected from a xylanase, a beta-glucanase, a cellulase and a xyloglucanase; and
f) a wild-type phytase and xylanase and further comprising enzymes suitable for insoluble and soluble arabinoxylans such as enzymes selected from a xylanase, a beta-glucanase, a cellulase and a xyloglucanase.

In one embodiment, the co-granule comprises at least two classes of enzymes, wherein the at least two classes of enzymes are selected from the group consisting of
a) a phytase having at least 80% sequence identity, such as at least 85%, at least 90%, at least 95%, such as at least 99% or 100% sequence identity to SEQ ID NO: 19 and a xylanase having at least 80% sequence identity, such as at least 85%, at least 90%, at least 95%, such as at least 99% or 100% sequence identity to SEQ ID NO:1;
b) an alpha-amylase (EC 3.2.1.1) having at least 80% sequence identity, such as at least 85%, at least 90%, at least 95%, such as at least 99% or 100% sequence identity to SEQ ID NO: 2 and a protease having at least 80% sequence identity, such as at least 85%, at least 90%, at least 95%, such as at least 99% or 100% sequence identity to SEQ ID NO:16;
c) an alpha-amylase (EC 3.2.1.1) having at least 80% sequence identity, such as at least 85%, at least 90%, at least 95%, such as at least 99% or 100% sequence identity to SEQ ID NO: 2 and a protease having at least 80% sequence identity, such as at least 85%, at least 90%, at least 95%, such as at least 99% or 100% sequence identity to SEQ ID NO:15;
d) a phytase having at least 80% sequence identity, such as at least 85%, at least 90%, at least 95%, such as at least 99% or 100% sequence identity to SEQ ID NO: 18 and a xylanase having at least 80% sequence identity, such as at least 85%, at least 90%, at least 95%, such as at least 99% or 100% sequence identity to SEQ ID NO:3, preferably further comprising one or more of a beta-glucanase, a cellulase, and a xyloglucanase, such as a beta-glucanase having at least 80% sequence identity, such as at least 85%, at least 90%, at least 95%, such as at least 99% or 100% sequence identity to SEQ ID NO:4; and such as a cellulase having at least 80% sequence identity, such as at least 85%, at least 90%, at least 95%, such as at least 99% or 100% sequence identity to SEQ ID NO:5; such as a xyloglucanase having at least 80% sequence identity, such as at least 85%, at least 90%, at least 95%, such as at least 99% or 100% sequence identity to SEQ ID NO:6;
e) a phytase having at least 80% sequence identity, such as at least 85%, at least 90%, at least 95%, such as at least 99% or 100% sequence identity to SEQ ID NO: 19 and a xylanase having at least 80% sequence identity, such as at least 85%, at least 90%, at least 95%, such as at least 99% or 100% sequence identity to SEQ ID NO:3, preferably further comprising one or more of a beta-glucanase, a cellulase, and a xyloglucanase, such as a beta-glucanase having at least 80% sequence identity, such as at least 85%, at least 90%, at least 95%, such as at least 99% or 100% sequence identity to SEQ ID NO:4; and such as a cellulase having at least 80% sequence identity, such as at least 85%, at least 90%, at least 95%, such as at least 99% or 100% sequence identity to SEQ ID NO:5; such as a xyloglucanase having at least 80% sequence identity, such as at least 85%, at least 90%, at least 95%, such as at least 99% or 100% sequence identity to SEQ ID NO:6.

In one embodiment, the co-granule comprises at least two classes of enzymes, wherein the at least two classes of enzymes are selected from the group consisting of
i. a phytase having at least 80% sequence identity, such as at least 85%, at least 90%, at least 95%, such as at least 99% or 100% sequence identity to SEQ ID NO: 18 and a xylanase having at least 80% sequence identity, such as at least 85%, at least 90%, at least 95%, such as at least 99% or 100% sequence identity to SEQ ID NO:1;
ii. a phytase having at least 80% sequence identity, such as at least 85%, at least 90%, at least 95%, such as at least 99% or 100% sequence identity to SEQ ID NO: 18 and a xylanase having at least 80% sequence identity, such as at least 85%, at least 90%, at least 95%, such as at least 99% or 100% sequence identity to SEQ ID NO:1;
iii. an amylase having at least 80% sequence identity, such as at least 85%, at least 90%, at least 95%, such as at least 99% or 100% sequence identity to SEQ ID NO:2 and a protease having at least 80% sequence identity, such as at least 85%, at least 90%, at least 95%, such as at least 99% or 100% sequence identity to SEQ ID NO:16;
iv. an endo 1,4-beta-xylanase having at least 80% sequence identity, such as at least 85%, at least 90%, at least 95%, such as at least 99% or 100% sequence identity to SEQ ID NO:3, optionally with an endo-1,3(4)-beta-glucanase having at least 80% sequence identity, such as at least 85%, at least 90%, at least 95%, such as at least 99% or 100% sequence identity to SEQ ID NO:4 and an endo 1,4-beta-glucanase having at least 80% sequence identity, such as at least 85%, at least 90%, at least 95%, such as at least 99% or 100% sequence identity to SEQ ID NO:4; and further comprising a protease having at least 80% sequence identity, such as at least 85%, at least 90%, at least 95%, such as at least 99% or 100% sequence identity to SEQ ID NO: 15;
v. an endo 1,4-beta-xylanase having at least 80% sequence identity, such as at least 85%, at least 90%, at least 95%, such as at least 99% or 100% sequence identity to SEQ ID NO: 3, an endo-1,3(4)-beta-glucanase and endo 1,4-beta-glucanase and a phytase having at least 80% sequence identity, such as at least 85%, at least 90%, at least 95%, such as at least 99% or 100% sequence identity to SEQ ID NO:18;
vi. an endo 1,4-beta xylanase having at least 80% sequence identity, such as at least 85%, at least 90%, at least 95%, such as at least 99% or 100% sequence identity to SEQ ID NO:3, an endo 1,4-beta xylanase xylanase having at least 80% sequence identity, such as at least 85%, at least 90%, at least 95%, such as at least 99% or 100% sequence identity to SEQ ID NO:4; an endo 1,3(4) beta-glucanase having at least 80% sequence identity, such as at least 85%, at least 90%, at least 95%, such as at least 99% or 100% sequence identity to SEQ ID NO:5; and a phytase having at least 80% sequence identity, such as at least 85%, at least 90%, at least 95%, such as at least 99% or 100% sequence identity to SEQ ID NO:19;
vii. a xylanase having at least 80% sequence identity, such as at least 85%, at least 90%, at least 95%, such as at least 99% or 100% sequence identity to SEQ ID NO:1 and a phytase of a phytase having at least 80% sequence identity, such as at least 85%, at least 90%, at least 95%, such as at least 99% or 100% sequence identity to SEQ ID NO:19;
viii. an endo 1,4-beta-xylanase, such as an endo 1,4-beta-xylanase having at least 80% sequence identity, such as at least 85%, at least 90%, at least 95%, such as at least 99% or 100% sequence identity to SEQ ID NO:3; an endo-1,3(4)-beta-glucanase, such as an endo-1,3(4)-beta-glucanase having at least 80% sequence identity, such as at least 85%, at least 90%, at least 95%, such as at least 99% or 100% sequence identity to SEQ ID NO:4, an endo 1,4-beta-glucanase, an xylanase, such as a xylanase having at least 80% sequence identity, such as at least 85%, at least 90%, at least 95%, such as at least 99% or 100% sequence identity to SEQ ID NO:1, and optionally a beta-glucanase, and/or an amylase, such as an amylase having at least 80% sequence identity, such as at least 85%, at least 90%, at least 95%, such as at least 99% or 100% sequence identity to SEQ ID NO:2; and
ix. a beta-glucanase, such as a beta-glucanase having at least 80% sequence identity such as at least 85%, at least 90%, at least 95%, such as at least 99% or 100% sequence identity to SEQ ID NO:8 and an amylase, such as an amylase having at least 80% sequence identity such as at least 85%, at least 90%, at least 95%, such as at least 99% or 100% sequence identity to SEQ ID NO:2; and a protease, such as a protease having at least 80% sequence identity such as at least 85%, at least 90%, at least 95%, such as at least 99% or 100% sequence identity to SEQ ID NO:15 or SEQ ID NO:16.

An aspect of the invention is directed to a granulate composition comprising co-granules, the co-granule comprising at least two classes of enzymes, said co-granules comprising a core unit and a coating, wherein the co-granule core comprises the at least two classes of enzymes, wherein the at least two classes of enzymes are selected from the group consisting of a phytase having at least 80% sequence identity, such as at least 85%, at least 90%, at least 95%, such as at least 99% or 100% sequence identity to SEQ ID NO: 18 and a xylanase having at least 80% sequence identity, such as at least 85%, at least 90%, at least 95%, such as at least 99% or 100% sequence identity to SEQ ID NO:1.

An aspect of the invention is directed to a granulate composition comprising co-granules, the co-granule comprising at least two classes of enzymes, said co-granules comprising a core unit and a coating, wherein the co-granule core comprises the at least two classes of enzymes, wherein the at least two classes of enzymes are selected from the group consisting of an amylase having at least 80% sequence identity, such as at least 85%, at least 90%, at least 95%, such as at least 99% or 100% sequence identity to SEQ ID NO:2 and a protease having at least 80% sequence identity, such as at least 85%, at least 90%, at least 95%, such as at least 99% or 100% sequence identity to SEQ ID NO:16.

An aspect of the invention is directed to a granulate composition comprising co-granules, the co-granule comprising at least two classes of enzymes, said co-granules comprising a core unit and a coating, wherein the co-granule core comprises the at least two classes of enzymes, wherein the at least two classes of enzymes are selected from the group consisting of an endo 1,4-beta-xylanase having at least 80% sequence identity, such as at least 85%, at least 90%, at least 95%, such as at least 99% or 100% sequence identity to SEQ ID NO:3, optionally with an endo-1,3(4)-beta-glucanase having at least 80% sequence identity, such as at least 85%, at least 90%, at least 95%, such as at least 99% or 100% sequence identity to SEQ ID NO:4 and an endo 1,4-beta-glucanase having at least 80% sequence identity, such as at least 85%, at least 90%, at least 95%, such as at least 99% or 100% sequence identity to SEQ ID NO:4; and further comprising a protease having at least 80% sequence identity, such as at least 85%, at least 90%, at least 95%, such as at least 99% or 100% sequence identity to SEQ ID NO: 15.

An aspect of the invention is directed to a granulate composition comprising co-granules, the co-granule comprising at least two classes of enzymes, said co-granules comprising a core unit and a coating, wherein the co-granule core comprises the at least two classes of enzymes, wherein the at least two classes of enzymes are selected from the group consisting of an endo 1,4-beta-xylanase having at least 80% sequence identity, such as at least 85%, at least 90%, at least 95%, such as at least 99% or 100% sequence identity to SEQ ID NO: 3, an endo-1,3(4)-beta-glucanase and endo 1,4-beta-glucanase and a phytase having at least 80% sequence identity, such as at least 85%, at least 90%, at least 95%, such as at least 99% or 100% sequence identity to SEQ ID NO:18.

An aspect of the invention is directed to a granulate composition comprising co-granules, the co-granule comprising at least two classes of enzymes, said co-granules comprising a core unit and a coating, wherein the co-granule core comprises the at least two classes of enzymes, wherein the at least two classes of enzymes are selected from the group consisting of an endo 1,4-beta xylanase having at least 80% sequence identity, such as at least 85%, at least 90%, at least 95%, such as at least 99% or 100% sequence identity to SEQ ID NO:3, an endo 1,4-beta xylanase xylanase having at least 80% sequence identity, such as at least 85%, at least 90%, at least 95%, such as at least 99% or 100% sequence identity to SEQ ID NO:4; an endo 1,3(4) beta-glucanase having at least 80% sequence identity, such as at least 85%, at least 90%, at least 95%, such as at least 99% or 100% sequence identity to SEQ ID NO:5; and a phytase having at least 80% sequence identity, such as at least 85%, at least 90%, at least 95%, such as at least 99% or 100% sequence identity to SEQ ID NO:19.

An aspect of the invention is directed to a granulate composition comprising co-granules, the co-granule comprising at least two classes of enzymes, said co-granules comprising a core unit and a coating, wherein the co-granule core comprises the at least two classes of enzymes, wherein the at least two classes of enzymes are selected from the group consisting of a xylanase having at least 80% sequence identity, such as at least 85%, at least 90%, at least 95%, such as at least 99% or 100% sequence identity to SEQ ID NO:1 and a phytase of a phytase having at least 80% sequence identity, such as at least 85%, at least 90%, at least 95%, such as at least 99% or 100% sequence identity to SEQ ID NO:19.

An aspect of the invention is directed to a granulate composition comprising co-granules, the co-granule comprising at least two classes of enzymes, said co-granules comprising a core unit and a coating, wherein the co-granule core comprises the at least two classes of enzymes, wherein the at least two classes of enzymes are selected from the group consisting of an endo 1,4-beta-xylanase, such as an endo 1,4-beta-xylanase having at least 80% sequence identity, such as at least 85%, at least 90%, at least 95%, such as at least 99% or 100% sequence identity to SEQ ID NO:3; an endo-1,3(4)-beta-glucanase, such as an endo-1,3(4)-beta-glucanase having at least 80% sequence identity, such as at least 85%, at least 90%, at least 95%, such as at least 99% or 100% sequence identity to SEQ ID NO:4, an endo 1,4-beta-glucanase, an xylanase, such as a xylanase having at least 80% sequence identity, such as at least 85%, at least 90%, at least 95%, such as at least 99% or 100% sequence identity to SEQ ID NO:1, and optionally a beta-glucanase, and/or an amylase, such as an amylase having at least 80% sequence identity, such as at least 85%, at least 90%, at least 95%, such as at least 99% or 100% sequence identity to SEQ ID NO:2.

An aspect of the invention is directed to a granulate composition comprising co-granules, the co-granule comprising at least two classes of enzymes, said co-granules comprising a core unit and a coating, wherein the co-granule core comprises the at least two classes of enzymes, wherein the at least two classes of enzymes are selected from the group consisting of a beta-glucanase, such as a beta-glucanase having at least 80% sequence identity such as at least 85%, at least 90%, at least 95%, such as at least 99% or 100% sequence identity to SEQ ID NO:8 and an amylase, such as an amylase having at least 80% sequence identity such as at least 85%, at least 90%, at least 95%, such as at least 99% or 100% sequence identity to SEQ ID NO:2; and a protease, such as a protease having at least 80% sequence identity such as at least 85%, at least 90%, at least 95%, such as at least 99% or 100% sequence identity to SEQ ID NO:15 or SEQ ID NO:16.

A composition comprising multiple co-granules of product 1 were prepared with high homogeneity of the enzymes so as to give the same protein load pr. g in the enzyme product, while having a better activity distribution in the feed when applied, compared to the single solutions.

The core comprises two or more enzymes, in the form of concentrated dry matter.

The core can either be
1. a homogeneous blend of two or more enzymes, or
2. an inert particle with two enzymes classes applied onto it, or
3. a homogenous blend of at least two enzyme classes, typically at least one carbohydrase, and materials which act as binders which are coated with the two or more enzyme classes.

In a preferred embodiment, the core comprises a carbohydrase and at least one other enzyme.

The core particle size of the present invention is in a particular embodiment 20-1900 μm. In a more particular embodiment of the present invention the core particle size is 50-1400 μm. In an even more particular embodiment of the present invention the core particle size is 150-1100 μm.

In a most particular embodiment of the present invention the core particle size is 250-1200 μm.

In the instances where the core comprises an inert particle, the inert particle may be water soluble or water insoluble, e.g. starch, e.g. in the form of cassava or wheat; or a sugar (such as sucrose or lactose), or a salt (such as sodium chloride or sodium sulphate). Suitable inert particle materials of the present invention include inorganic salts, sugars, sugar alcohols, small organic molecules such as organic acids or salts, minerals such as clays or silicates or a combination of two or more of these. Inert particles may be produced by a variety of granulation techniques including: crystallisation, precipitation, pan-coating, fluid bed coating, fluid bed agglomeration, rotary atomization, extrusion, prilling, spheronization, size reduction methods, drum granulation, and/or high shear granulation.

Suitable particle sizes of the co-granule of the present invention are found to be 50-2000 μm, more particularly 100-1500 μm. In an embodiment of the invention, the particle size of the co-granule is more than 250 μm. In a further embodiment of the invention, the particle size is below 1200 μm. In yet a further embodiment, the particle size is between 250-1200 μm. In another embodiment of the present invention the particle size of the finished co-granule is 250-900 μm. In yet another embodiment of the present invention the mean particle size of the finished co-granule is 500-700 μm. In still another embodiment of the present invention the particle size of the finished co-granule is 600-1200 μm. In still another embodiment of the present invention, the particle size of the finished co-granule is 600-900 μm.

In the instances where the core comprises one or more binders, the binders may be synthetic polymers such as e.g. a vinyl polymer, polyvinyl pyrrolidone (PVP), polyvinyl alcohol (PVA), polyvinyl acetate, polyacrylate, polymethacrylate, poly-acrylamide, polysulfonate, polycarboxylate, and copolymers thereof, waxes including fats, fermentation broth, carbohydrates, salts or polypeptides. In a particular embodiment, the binder is a polypeptide. The polypeptide may be selected from gelatin, collagen, casein, chitosan, poly aspartic acid and poly glutamatic acid. In another particular embodiment the binder is a cellulose derivative such as hydroxypropyl cellulose, methyl cellulose or CMC. A suitable binder is a carbohydrate binder such as dextrin e.g Glucidex 21D or Avedex W80.

In one embodiment, the core may comprise a salt. The salt may be an inorganic salt, e.g. a salt of sulfate, sulfite, phosphate, phosphonate, nitrate, chloride or carbonate or salts of simple organic acids (less than 10 carbon atoms e.g. 6 or less carbon atoms) such as citrate, malonate or acetate. Examples of cations in these salt are alkali or earth alkali metal ions, although the ammonium ion or metal ions of the first transition series, such as sodium, potassium, magnesium, calcium, zinc or aluminium. Examples of anions include chloride, iodide, sulfate, sulfite, bisulfite, thiosulfate, phosphate, monobasic phosphate, dibasic phosphate, hypophosphite, dihydrogen pyrophosphate, carbonate, bicarbonate, metasilicate, citrate, malate, maleate, malonate, succinate, lactate, formate, acetate, butyrate, propionate, benzoate, tartrate, ascorbate or gluconate. In particular alkali- or earth alkali metal salts of sulfate, sulfite, phosphate, phosphonate, nitrate, chloride or carbonate or salts of simple organic acids such as citrate, malonate or acetate may be used. Specific examples include NaH₂PO₄, Na₂HPO₄, Na₃PO₄, (NH₄)H₂PO₄, K₂HPO₄, KH₂PO₄, Na₂SO₄, K₂SO₄, KHSO₄, ZnSO₄, MgSO₄, CuSO₄, Mg(NO₃)₂, (NH₄)₂SO₄, sodium borate, magnesium acetate and sodium citrate. The salt in the core of the particle may also be a hydrated salt, i.e. a crystalline salt hydrate with bound water(s) of crystallization, such as described in WO 99/32595. Examples of hydrated salts include magnesium sulfate heptahydrate (MgSO₄(7H₂O)), zinc sulfate heptahydrate (ZnSO₄(7H₂O)), sodium phosphate dibasic heptahydrate (Na₂HPO₄(7H₂O)), magnesium nitrate hexahydrate (Mg(NO₃)₂(6H₂O)), sodium borate decahydrate, sodium citrate dihydrate and magnesium acetate tetrahydrate.

In one embodiment, the core and/or the salt layer may comprise a moisture absorbing compound. The moisture absorbing compound serves as a buffer which is able to decrease water activity by reducing free water in contact with the muramidase in the co-granule. If the moisture absorbing compound is added to the core, it is important that there is excessive buffer capacity to remove the water present after application of the salt layer. In one embodiment, the moisture absorbing compound has a water uptake of more than 3%, such as more than 5%, such as more than 10% water uptake. The water uptake is found as the equilibrium water uptake at 25° C. and 70% relative humidity after one week. The amount of moisture absorbing compound added to the co-granule is more than 1%, more than 2%, more than 5%, or more than 10% w/w of the co-granule.

The moisture absorbing compound may be either organic or inorganic compounds and may be selected from, but is not limited to, the group consisting of flour, starch, corn cob products, cellulose and silica gel.

The co-granule may comprise additional materials such as process aids, fillers, fibre materials, stabilizing agents, solubilising agents, suspension agents, viscosity regulating agents, light spheres, plasticizers, salts, lubricants and fragrances. Process aids may e.g. be provided as powdering and may e.g. be CaCO₃, talcum and/or kaolin. Suitable fillers are water soluble and/or insoluble inorganic salts such as finely ground alkali sulphate, alkali carbonate and/or alkali chloride, clays such as kaolin (e.g. SPESWHITE™, English China Clay), bentonites, talcs, zeolites, chalk, calcium carbonate and/or silicates. Typical fillers are di-sodium sulphate and calcium-lignosulphonate. Stabilising or protective agents are such as conventionally used in the field of granulation. Stabilising or protective agents may fall into several categories: alkaline or neutral materials, reducing agents, antioxidants and/or salts of first transition series metal ions.

Each of these may be used in conjunction with other protective agents of the same or different categories. Examples of alkaline protective agents are alkali metal silicates, carbonates or bicarbonates. Examples of reducing protective agents are salts of sulfite, thiosulfite, thiosulfate or MnSO₄ while examples of antioxidants are methionine, butylated hydroxytoluene (BHT) or butylated hydroxyanisol (BHA). In particular stabilising agents may be salts of thiosulfates, e.g. sodium thiosulfate or methionine. Still other examples of useful stabilizers are gelatine, urea, sorbitol, glycerol, casein, Poly vinyl pyrrolidone (PVP), hydroxypropylmethylcellulose (HPMC), carboxymethyl cellulose (CMC), hydroxyethylcellulose (HEC), powder of skimmed milk and/or edible oils, such as soy oil or canola oil. Particular stabilizing agents in feed co-granules are a lactic acid source or starch. A preferred lactic acid source is corn steep liquor. It is also well known in the art that enzyme substrates such as starch, lipids, proteins etc can act as stabilizers for enzymes

The coating typically comprises a salt layer. The salt layer, may in a particular embodiment of the present invention contribute between 20-99% w/w of the co-granule, such as between 20-70% w/w, 30-60% w/w, 40-60% w/w or 50-60% w/w of the co-granule. In one embodiment, the salt layer. In one embodiment the salt layer comprises at least 60% w/w, e.g. 65% w/w or 70% w/w salt, which may be at least 75% w/w, e.g. at least 80% w/w, at least 85% w/w, e.g. at least 90% w/w or at least 95% w/w, even at least 99% w/w salt.

In a particular embodiment of the present invention the amount of salt in the salt layer of the co-granule constitutes at least 40% w/w of the salt layer.

In a particular embodiment of the present invention the amount of salt in the salt layer of the co-granules in the feed composition, such as e.g. stem treated feed compositions, constitutes at least 40% w/w of the salt layer.

In a particular embodiment of the present invention the amount of salt in the salt layer of the co-granules to be used for feed compositions, such as e.g. stem treated feed compositions, constitutes at least 40% w/w of the salt layer.

In a further particular embodiment of the present invention the amount of salt in the salt layer of the co-granule constitutes at least 50% w/w of the salt layer.

In a further particular embodiment of the present invention the amount of salt in the salt layer of the co-granules in the feed composition, such as e.g. stem treated feed compositions, constitutes at least 50% w/w of the salt layer.

In a further particular embodiment of the present invention the amount of salt in the salt layer of the co-granules to be used for feed compositions, such as e.g. stem treated feed compositions, constitutes at least 50% w/w of the salt layer.

In a yet further particular embodiment of the present invention the amount of salt in the salt layer of the co-granule constitutes at least 60% w/w of the salt layer.

In a yet further particular embodiment of the present invention the amount of salt in the salt layer of the co-granules in the feed composition, such as e.g. stem treated feed compositions, constitutes at least 60% w/w of the salt layer.

In a yet further particular embodiment of the present invention the amount of salt in the salt layer of the co-granules to be used for feed compositions, such as e.g. stem treated feed compositions, constitutes at least 60% w/w of the salt layer.

To be able to provide acceptable protection, the salt layer preferably has a certain thickness. In a particular embodiment of the present invention the salt layer is at least 15 μm thick. In a more particular embodiment, the thickness of the salt layer is at least 22 μm. In an even more particular embodiment, the total thickness of the salt layer is at least 30 μm. In a most particular embodiment, the total thickness of the salt layer is at least 37 μm. In a most particular embodiment, the total thickness of the salt layer is at least 45 μm. In a most particular embodiment, the total thickness of the salt layer is at least 52 μm. In a particular embodiment of the present invention the thickness of the salt layer is below 100 μm. In a more particular embodiment, the thickness of the salt layer is below 60 μm. In an even more particular embodiment, the total thickness of the salt layer is below 40 μm.

In a particular embodiment of the present invention the thickness of the salt layer of the co-granule of the present invention is at least 30 μm. In another particular embodiment of the present invention the thickness of the salt layer of the co-granule of the present invention is at least 37 μm. In yet another particular embodiment of the present invention the thickness of the salt layer of the co-granule of the present invention is at least 45 μm.

In a particular embodiment of the present invention the thickness of the salt layer of the co-granules to be used for feed composition, such as e.g. the steam treated pelletized feed composition, is at least 30 μm. In another particular embodiment of the present invention the thickness of the salt layer of the co-granules to be used for feed composition, such as e.g. the steam treated pelletized feed composition, is at least 37 μm. In yet another particular embodiment of the present invention the thickness of the salt layer of the co-granules to be used for feed composition, such as e.g. the steam treated pelletized feed composition, is at least 45 μm.

In a particular embodiment of the present invention the thickness of the salt layer of the co-granules to be used for feed compositions, such as e.g. the steam treated pelletized feed compositions, is at least 30 μm. In another particular embodiment of the present invention the thickness of the salt layer of the co-granules to be used for feed compositions, such as e.g. the steam treated pelletized feed compositions, is at least 37 μm. In yet another particular embodiment of the present invention the thickness of the salt layer of the co-granules to be used for feed compositions, such as e.g. the steam treated pelletized feed compositions, is at least 45 μm.

In one embodiment the coated co-granule is a co-granule according to WO 01/25412, where the ratio between the diameter of the coated co-granule and the diameter of the core unit (abbreviated DG/DC) for this type of co-granules is at least 1.1, particularly at least 1.5, more particularly at least 2, more particularly at least 2.5, more particularly at least 3, most particularly at least 4. DG/DC is however particularly below about 100, particularly below about 50, more particularly below 25, and most particularly below 10. A particularly suitable range for DG/DC is about 4 to about 6. Thus, for such co-granules the thickness of the salt layer should be at least 25 μm. A particular thickness is at least 50 μm such as at least 75 μm, at least 100 μm, at least 150 μm, at least 200 μm, at least 250 μm or particularly at least 300 μm. The thickness of this kind of salt layer is usually below 800 μm. A particular thickness is below 500 μm such as below 350 μm, below 300 μm, below 250 μm, below 200 μm, below 150 μm or particularly below 80 μm.

The salt layer should encapsulate the core unit by forming a substantially continuous layer, i.e. as a salt layer having few or no holes, so that the core unit it is encapsulating has few or no uncoated areas. The salt layer should in particular be homogenous in thickness.

The salt to be added is preferably in the form of a salt solution or a salt suspension wherein the fine particles is less than 5 μm, such as less than 1 μm.

In a particular embodiment of the present invention, it is preferred to use a solution of salt as salt layer, but if the used salts have low solubility it can be preferable to use a suspension of salt instead of a solution, to be able to add more salt pr. litre liquid added to the co-granules. In a particular embodiment of the present invention the salt layer is prepared in accordance with the coating in WO 03/55967.

Referring to the salt in the salt layer it can either be one particular salt or a mixture of salts. The salt used may be an inorganic salt, e.g. salts of sulfate, sulfite, phosphate, phosphonate, nitrate, chloride or carbonate or salts of simple organic acids (less than 10 carbon atoms e.g. 6 or less carbon atoms) such as citrate, malonate or acetate. Examples of cations in these salts are alkali or earth alkali metal ions, although the ammonium ion or metal ions of the first transition series, such as sodium, potassium, magnesium, calcium, zinc or aluminium. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, lactate, formate, acetate, butyrate, propionate, benzoate, tartrate, ascorbate or gluconate. In particular alkali- or earth alkali metal salts of sulfate, sulfite, phosphate, phosphonate, nitrate, chloride or carbonate or salts of simple organic acids such as citrate, malonate or acetate may be used. Specific examples include NaH₂PO₄, Na₂HPO₄, Na₃PO₄, (NH₄)H₂PO₄, K₂HPO₄, KH₂PO₄, Na₂SO₄, K₂SO₄, KHSO₄, ZnSO₄, MgSO₄, CuSO₄, Mg(NO₃)₂, (NH₄)₂SO₄, sodium borate, magnesium acetate and sodium citrate.

The salt may also be a hydrated salt, i.e. a crystalline salt hydrate with bound water(s) of crystallization, such as described in WO 99/32595. Examples of hydrated salts include magnesium sulfate heptahydrate (MgSO₄(7H₂O)), zinc sulfate heptahydrate (ZnSO₄(7H₂O)), sodium phosphate dibasic heptahydrate (Na₂HPO₄(7H₂O)), magnesium nitrate hexahydrate (Mg(NO₃)₂(6H₂O)), sodium borate decahydrate, sodium citrate dihydrate and magnesium acetate tetrahydrate.

In an embodiment of the present invention the salt layer does not comprise a hydrated salt. In a particular embodiment of the present invention the salt layer does not comprise a salt comprising more than four water molecules at 50° C.

In a particular embodiment of the present invention the Specific examples of suitable salts of the invention are NaCl (CH_{20° C.}=76%), Na₂CO₃ (CH_{20° C.}=92%), NaNO₃ (CH_{20° C.}=73%), Na₂HPO₄ (CH_{20° C.}=95%), Na₃PO₄ (CH_{25° C.}=92%), NH₄Cl (CH_{20° C.}=79.5%), (NH₄)₂HPO₄ (CH_{20° C.}=93.0%), NH₄H₂PO₄ (CH_{20° C.}=93.1%), (NH₄)₂SO₄ (CH_{20° C.}=81.1%), KCl (CH_{20° C.}=85%), K₂HPO₄ (CH_{20° C.}=92%), KH₂PO₄ (CH_{20° C.}=96.5%), KNO₃ (CH_{20° C.}=93.5%), Na₂SO₄ (CH_{20° C.}=93%), K₂SO₄(CH_{20° C.}=98%), KHSO₄ (CH_{20° C.}=86%), MgSO₄ (CH_{20° C.}=90%), ZnSO₄ (CH_{20° C.}=90%) and sodium citrate (CH_{25° C.}=86%).

In a particular embodiment of the present invention the salt is selected from the group consisting of NaCl, Na₂CO₃, NaNO₃, Na₂HPO₄, Na₃PO₄, NH₄Cl, (NH₄)₂HPO₄, NH₄H₂PO₄, (NH₄)₂SO₄, KCl, K₂HPO₄, KH₂PO₄, KNO₃, Na₂SO₄, K₂SO₄, KHSO₄, MgSO₄, ZnSO₄, NaCl and sodium citrate or mixtures thereof. In a more particular embodiment of the present invention the salt is selected from the group consisting of NaCl, Na₂CO₃, NaNO₃, Na₂HPO₄, Na₃PO₄, NH₄Cl, (NH₄)₂HPO₄, NH₄H₂PO₄, (NH₄)₂SO₄, KCl, K₂HPO₄, KH₂PO₄, KNO₃, Na₂SO₄, K₂SO₄, KHSO₄, NaCl and sodium citrate or mixtures thereof.

In a particular embodiment of the present invention the salt comprised in the salt layer of the co-granule of the present invention is selected from the group consisting of NaCl, Na₂CO₃, NaNO₃, Na₂HPO₄, Na₃PO₄, NH₄Cl, (NH₄)₂HPO₄, NH₄H₂PO₄, (NH₄)₂SO₄, KCl, K₂HPO₄, KH₂PO₄, KNO₃, Na₂SO₄, K₂SO₄, KHSO₄, MgSO₄, ZnSO₄, NaCl and sodium citrate or mixtures thereof.

In a particular embodiment of the present invention the salt comprised in the salt layer of the co-granule of the steam treated pelletized feed composition is selected from the group of NaCl, Na₂CO₃, NaNO₃, Na₂HPO₄, Na₃PO₄, NH₄Cl, (NH₄)₂HPO₄, NH₄H₂PO₄, (NH₄)₂SO₄, KCl, K₂HPO₄, KH₂PO₄, KNO₃, Na₂SO₄, K₂SO₄, KHSO₄, MgSO₄, ZnSO₄, NaCl and sodium citrate or mixtures thereof.

In a particular embodiment of the present invention the salt comprised in the salt layer of the the co-granules to be used for steam treated pelletized feed compositions is selected from the group of NaCl, Na₂CO₃, NaNO₃, Na₂HPO₄, Na₃PO₄, NH₄Cl, (NH₄)₂HPO₄, NH₄H₂PO₄, (NH₄)₂SO₄, KCl, K₂HPO₄, KH₂PO₄, KNO₃, Na₂SO₄, K₂SO₄, KHSO₄, MgSO₄, ZnSO₄, NaCl and sodium citrate or mixtures thereof.

### A Hydrophobic Layer

The granule may comprise an outer hydrophobic layer. The outer hydrophobic layer, may in a particular embodiment of the present invention contribute between 1-10% w/w of the co-granule, such as between 1-5% w/w, or between 2-3% w/w of the co-granule. In one embodiment, a hydrophobic layer contributes about 2% w/w of the co-granule. In another embodiment, a hydrophobic layer contributes about 3% w/w of the co-granule.

In a particular embodiment of the present invention the amount of hydrophobic coating material in a hydrophobic layer of the co-granule constitutes at least 60% w/w of a hydrophobic layer.

In a particular embodiment of the present invention the amount of hydrophobic coating material in a hydrophobic layer of the co-granules in the feed composition, such as e.g. stem treated feed compositions, constitutes at least 60% w/w of the outer hydrophobic layer.

In a particular embodiment of the present invention the amount of hydrophobic coating material in a hydrophobic layer of the co-granules to be used for feed compositions, such as e.g. stem treated feed compositions, constitutes at least 60% w/w of the outer hydrophobic layer.

To be able to provide acceptable protection, a hydrophobic layer preferably has a certain thickness. In a particular embodiment of the present invention a hydrophobic layer is at least 1 μm thick. In a more particular embodiment the thickness of a hydrophobic layer is at least 1.5 μm.

In an even more particular embodiment the total thickness of a hydrophobic layer is at least 2 μm.

In a most particular embodiment the total thickness of a hydrophobic layer is at least 4 μm. In a most particular embodiment the total thickness of a hydrophobic layer is at least 7 μm. In a particular embodiment of the present invention the thickness of a hydrophobic layer is below 10 μm. In a more particular embodiment the thickness of a hydrophobic layer is below 7 μm. In an even more particular embodiment the total thickness of a hydrophobic layer is below 4 μm.

In a particular embodiment of the present invention the thickness of a hydrophobic layer of the co-granule of the present invention is at least 1.5 μm. In another particular embodiment of the present invention the thickness of a hydrophobic layer of the co-granule of the present invention is at least 2 μm.

In a particular embodiment of the present invention the thickness of a hydrophobic layer of the co-granules to be used for feed compositions, such as e.g. the steam treated pelletized feed composition, is at least 1.5 μm. In another particular embodiment of the present invention the thickness of a hydrophobic layer of the co-granules to be used for feed compositions, such as e.g. the steam treated pelletized feed composition, is at least 2 μm.

In a particular embodiment of the present invention the thickness of a hydrophobic layer of the co-granules to be used for feed compositions, such as e.g. steam treated pelletized feed compositions, is at least 1.5 μm. In another particular embodiment of the present invention the thickness of a hydrophobic layer of the co-granules to be used for feed compositions, such as e.g. steam treated pelletized feed compositions, is at least 2 μm.

A hydrophobic layer should encapsulate the salt layer by forming a substantially continuous layer, i.e. an outer hydrophobic layer having few or no holes, so that the salt layer it is encapsulating has few or no uncoated areas. A hydrophobic layer should in a preferred embodiment be homogenous in thickness.

Referring to the hydrophobic coating material in a hydrophobic layer it can either be one particular hydrophobic coating material or a mixture of hydrophobic coating materials.

The hydrophobic coating material may include oils and/or waxes, including, without limitations, hydrogenated vegetable oils such as hydrogenated castor oil, hydrogenated palm kernel oil, hydrogenated palm oil, hydrogenated cotton seeds, hydrogenated soy bean oil and/or hydrogenated rapeseed oil, a blend of hydrogenated and unhydrogenated vegetable oil, 12-hyroxystearic acid, microcrystalline wax such as Cerit HOT, and high-melting paraffin waxes such as Mekon White.

Further hydrophobic coating materials included in the invention are combinations with water immiscible liquids or low melting point hydrophobic solids that produce a mixture with a reduced melting point. These include waxes, C26 and higher, paraffin waxes, cholesterol, fatty alcohols, such as cetyl alcohol, mono-, di- and/or triglycerides of animal and vegetable origin such as hydrogenated ox tallow, hydrogenated fat, hydrogenated castor oil, fat derivatives such as fatty acids, soaps, esters, hydrophobic starches such as ethyl cellulose, lecithin. The waxes may be of natural origin, meaning they may be animal, vegetable or mineral. Animal waxes include, without limitation, beeswax, lanolin, shellac wax and Chinese insect wax. Vegetable wax includes, without limitation, carnauba, candelilla, bayberry and sugar cane waxes. Mineral waxes include, without limitation, fossil or earth waxes including ozokerite, ceresin and montan or petroleum waxes, including paraffin and microcrystalline waxes. Alternatively the waxes may be synthetic or mixtures of natural and synthetic waxes. For example, synthetic or mixtures of natural and synthetic waxes may include low molecular weight partially oxidized polyethylene, which may be preferentially co-melted with paraffin. The fatty derivatives may be either fatty acids, fatty acid amides, fatty alcohols, fatty esters or mixtures of these. The acid amide may be stearamide. Sterols or long chain sterol esters may also be such as cholesterol or ergosterol.

A preferred hydrophobic coating material is palm oil or hydrogenated palm oil.

The present invention is also directed to methods for the preparation of an enzyme-enriched animal feed comprising the co-granules of the invention, as well as to animal feed and feed additives comprising the co-granules of the invention.

As aspect of the invention is directed to an animal feed comprising an animal feed additive, said additive comprising co-granules, each co-granule comprising at least two classes of enzymes, said co-granules comprising a core unit and a protective coating. Preferably, the feed is prepared by adding co-granulate composition of the invention to feed stuff, the co-granulate composition comprising co-granules having a core which comprises the at least two classes of enzymes, wherein the granulate composition has a homogeneity such that i) the quantity of each enzyme in one co-granule is 80-120% of the quantity of the same enzyme in at least 90% of the co-granules in said composition; and/or ii) wherein the ratio of the at least two classes of enzymes within a co-granule is 80% to 120% the ratio of the same two enzymes within at least 90% of the co-granules in said composition.

An aspect of the invention is directed to an animal feed comprising an animal feed additive, said additive comprising co-granules, each co-granule comprising at least two classes of enzymes, said co-granules comprising a core unit and a protective coating, wherein the co-granule core comprises the at least classes of enzymes, wherein the granulate composition has a homogeneity such that i) the quantity of each enzyme in one co-granule is 80-120% of the quantity of the same enzyme in at least 90% of the co-granules in said composition; and/or ii) wherein the ratio of the at least two classes of enzymes within a co-granule is 80% to 120% the ratio of the same two enzymes within at least 90% of the co-granules in said composition.

The invention is further directed to a method for manufacturing a feed composition comprising the steps of: i. mixing feed components with co-granules comprising a core and a coating wherein the core comprises at least two enzyme classes, (ii) steam treating said composition (ii), and pelleting said composition, characterized in that the coating comprises at least 60% w/w of a salt, wherein the salt is Na₂SO₄, and wherein the co-granules do not further comprise a wax coating.

An aspect of the invention is directed to a method for manufacturing a feed composition comprising the steps of:
i. mixing feed components with co-granules comprising a core and a coating, wherein the core comprises at least two enzyme classes,
ii. steam treating said composition (i), and
iii. pelleting said composition (ii),
   characterized in that the coating comprises a salt which has a constant humidity at 20° C. which is above 60%, and that the coating constitutes 20-80% w/w of the coated co-granules.

In particular embodiments, the co-granules of the invention are for use in feed for (i) non-ruminant animals; preferably (ii) mono-gastric animals; more preferably (iii) pigs, poultry, fish, and crustaceans; or, most preferably, (iv) pigs and poultry.

The co-granules of the invention can be fed to the animal before, after, or simultaneously with the diet. The latter is preferred. The co-granules can be added to a pre-mix or to an animal feed.

The term feed, feed composition, or diet means any compound, preparation, mixture, or composition suitable for, or intended for intake by an animal. More information about animal feed compositions is found below.

In another particular embodiment, the vegetable protein source is material from one or more plants of the family Chenopodiaceae, e.g. beet, sugar beet, spinach or quinoa.

Other examples of vegetable protein sources are rapeseed, and cabbage.

Soybean is a preferred vegetable protein source.

Other examples of vegetable protein sources are cereals such as barley, wheat, rye, oat, maize (corn), rice, and sorghum.

In still further particular embodiments, the animal feed composition of the invention contains 0-80% maize; and/or 0-80% sorghum; and/or 0-70% wheat; and/or 0-70% Barley; and/or 0-30% oats; and/or 0-40% soybean meal; and/or 0-10% fish meal; and/or 0-20% whey.

Suitable animal feed additives are enzyme inhibitors, fat soluble vitamins, water soluble vitamins, trace minerals and macro minerals.

In one embodiment, the present invention relates to an animal feed comprising a co-granule of the invention. The co-granules of the invention provide additional protein digestibility on top of endogenous proteases, resulting in a 3-6% increase in amino acid digestibility. The co-granules of the invention increase energy (ME) by at least 25 kcal/kg diet.

The co-granules contribute to sustainable poultry production by supporting:
1. efficient use of natural resources: lower soy utilization and more alternative (local and by-products) raw materials
2. reduction of livestock emissions: low Nitrogen emissions by enabling lower Crude Protein diets
3. lifetime performance & animal welfare: significantly reducing foot-pad lesions

Specifically developed for inclusion in animal diets, the enzymes of the invention significantly increases the protein digestion. They complement naturally occurring enzymes, and considerably increases nutrient supply as so to enhance animal performance. The co-granules improves the digestibility of a wide range of protein sources and cereals, allowing savings in feed costs in a reliable manner.

In a further aspect, the present invention relates to an animal feed additive, comprising a co-granule of the invention and one or more additional components selected from the group consisting of: one or more vitamins; one or more minerals; one or more amino acids; one or more phytogenics; one or more prebiotics; one or more organic acids; and one or more other feed ingredients. The following are non-exclusive lists of examples of these components:

Examples of fat-soluble vitamins are vitamin A, vitamin D3, vitamin E, and vitamin K, e.g. vitamin K3. Examples of water-soluble vitamins are vitamin B12, biotin and choline, vitamin B1, vitamin B2, vitamin B6, niacin, folic acid and panthothenate, e.g. Ca-D-panthothenate. Examples of trace minerals are manganese, zinc, iron, copper, iodine, selenium, and cobalt. Examples of macro minerals are calcium, phosphorus and sodium. Examples of amino acids which are used in animal feed are lysine, alanine, beta-alanine, threonine, methionine and tryptophan.

Phytogenics are a group of natural growth promoters or non-antibiotic growth promoters used as feed additives, derived from herbs, spices or other plants. Phytogenics can be single substances prepared from essential oils/extracts, essential oils/extracts, single plants and mixture of plants (herbal products) or mixture of essential oils/extracts/plants (specialized products).

Examples of phytogenics are rosemary, sage, oregano, thyme, clove, and lemongrass. Examples of essential oils are thymol, eugenol, meta-cresol, vaniline, salicylate, resorcine, guajacol, gingerol, lavender oil, ionones, irone, eucalyptol, menthol, peppermint oil, alpha-pinene; limonene, anethol, linalool, methyl dihydrojasmonate, carvacrol, propionic acid/propionate, acetic acid/acetate, butyric acid/butyrate, rosemary oil, clove oil, geraniol, terpineol, citronellol, amyl and/or benzyl salicylate, cinnamaldehyde, plant polyphenol (tannin), turmeric and curcuma extract.

Organic acids (C1-C7) are widely distributed in nature as normal constituents of plants or animal tissues. They are also formed through microbial fermentation of carbohydrates mainly in the large intestine. They are often used in swine and poultry production as a replacement of antibiotic growth promoters since they have a preventive effect on the intestinal problems like necrotic enteritis in chickens and Escherichia coli infection in young pigs. Organic acids can be sold as mono component or mixtures of typically 2 or 3 different organic acids. Examples of organic acids are propionic acid, formic acid, citric acid, lactic acid, sorbic acid, malic acid, acetic acid, fumaric acid, benzoic acid, butyric acid and tartaric acid or their salt (typically sodium or potassium salt such as potassium diformate or sodium butyrate).

Further, optional, feed-additive ingredients are colouring agents, e.g. carotenoids such as beta-carotene, astaxanthin, and lutein; aroma compounds; stabilisers; antimicrobial peptides; polyunsaturated fatty acids; reactive oxygen generating species; and/or at least one other enzyme selected from amongst another pectinase (EC 3.2.1.8); and/or beta-glucanase (EC 3.2.1.4 or EC 3.2.1.6).

Examples of antimicrobial peptides (AMP's) are CAP18, Leucocin A, Tritrpticin, Protegrin-1, Thanatin, Defensin, Lactoferrin, Lactoferricin, and Ovispirin such as Novispirin (Robert Lehrer, 2000), Plectasins, and Statins, including the compounds and polypeptides disclosed in WO 03/044049 and WO 03/048148, as well as variants or fragments of the above that retain antimicrobial activity.

Examples of antifungal polypeptides (AFP's) are the Aspergillus giganteus, and Aspergillus niger peptides, as well as variants and fragments thereof which retain antifungal activity, as disclosed in WO 94/01459 and WO 02/090384.

Examples of polyunsaturated fatty acids are C18, C20 and C22 polyunsaturated fatty acids, such as arachidonic acid, docosohexaenoic acid, eicosapentaenoic acid and gamma-linoleic acid, Examples of reactive oxygen generating species are chemicals such as perborate, persulphate, or percarbonate; and enzymes such as an oxidase, an oxygenase or a syntethase.

Usually fat- and water-soluble vitamins, as well as trace minerals form part of a so-called premix intended for addition to the feed, whereas macro minerals are usually separately added to the feed. A premix enriched with a co-granule of the invention is an example of an animal feed additive of the invention.

The nutritional requirements of these components (exemplified with poultry and piglets/pigs) are listed in Table A of WO 01/58275. Nutritional requirement means that these components should be provided in the diet in the concentrations indicated.

In the alternative, the animal feed additive of the invention comprises at least one of the individual components specified in Table A of WO 01/58275. At least one means either of, one or more of, one, or two, or three, or four and so forth up to all thirteen, or up to all fifteen individual components. More specifically, this at least one individual component is included in the additive of the invention in such an amount as to provide an in-feed-concentration within the range indicated in column four, or column five, or column six of Table A.

Animal feed compositions or diets have a relatively high content of protein. Poultry and pig diets can be characterised as indicated in Table B of WO 01/58275, columns 2-3. Fish diets can be characterised as indicated in column 4 of this Table B. Furthermore such fish diets usually have a crude fat content of 200-310 g/kg.

An animal feed composition according to the invention has a crude protein content of 50-800 g/kg (preferably 50-600 g/kg, more preferably 60-500 g/kg, even more preferably 70-500, and most preferably 80-400 g/kg) and furthermore comprises co-granules as defined herein. In additional preferred embodiments, the crude protein content is 150-800, 160-800, 170-800, 180-800, 190-800, or 200-800—all in g/kg (dry matter). Furthermore, or in the alternative (to the crude protein content indicated above), the animal feed composition of the invention has a content of metabolisable energy of 10-30 MJ/kg; and/or a content of calcium of 0.1-200 g/kg; and/or a content of available phosphorus of 0.1-200 g/kg; and/or a content of methionine of 0.1-100 g/kg; and/or a content of methionine plus cysteine of 0.1-150 g/kg; and/or a content of lysine of 0.5-50 g/kg. In particular embodiments, the content of metabolisable energy, crude protein, calcium, phosphorus, methionine, methionine plus cysteine, and/or lysine is within any one of ranges 2, 3, 4 or 5 in Table B of WO 01/58275 (R. 2-5). Crude protein is calculated as nitrogen (N) multiplied by a factor 6.25, i.e. Crude protein (g/kg)=N (g/kg)×6.25. The nitrogen content is determined by the Kjeldahl method (A.O.A.C., 1984, Official Methods of Analysis 14th ed., Association of Official Analytical Chemists, Washington DC).

The dietary content of calcium, available phosphorus and amino acids in complete animal diets is calculated on the basis of feed tables such as Veevoedertabel 1997, gegevens over chemische samenstelling, verteerbaarheid en voederwaarde van voedermiddelen, Central Veevoederbureau, Runderweg 6, 8219 pk Lelystad. ISBN 90-72839-13-7. Metabolisable energy can be calculated on the basis of the NRC publication Nutrient requirements in swine, ninth revised edition 1988, subcommittee on swine nutrition, committee on animal nutrition, board of agriculture, national research council. National Academy Press, Washington, D.C., pp. 2-6, and the European Table of Energy Values for Poultry Feed-stuffs, Spelderholt centre for poultry research and extension, 7361 DA Beekbergen, The Netherlands. Grafisch bedrijf Ponsen & looijen bv, Wageningen. ISBN 90-71463-12-5.

In a further aspect, the present invention relates to a method of improving the Average Metabolizable Energy, Food Conversion Ratio or Body Weight Gain of plant-based diet in a monogastric animal comprising administering an animal feed additive of the present invention or the animal feed of the present invention.

The present invention is further described by the following examples which should not be construed as limiting the scope of the invention.

### EXAMPLES

### Example 1—Preparation of the Granulate

Co-granule 1, co-granule comprising a core comprising a carbohydrase, and a salt layer on the core:
A powder mixture with the following composition
960 g cellulose, Arbocel BC200
600 g dextrin, Kaolin
ca.2000 g spray dried carbohydrase powder
ca. 500 g spray dried second enzyme/carbohydrase powder
7552 g ground Na₂SO₄
was granulated in a Lödige mixer FM 50 with a granulation fluid consisting of
480 g Sucrose
2220 g water

The granulation was carried out as described in U.S. Pat. No. 4,106,991, example 1.

The granulate was dried in a fluid bed dryer to a water content of less than 1% and sifted to obtain a product with particle size between 250 and 1200 micrometers.

4.0 kg of above-described co-granule cores were placed into a MP1 fluid bed.

The following mixture was prepared for applying a salt layer on the cores:
1600 g Na₂SO₄
4000 g water

The following bed set-up was used during coating:
Airflow: 215 m³/hour
Inlet temperature: 90° C.
Product temperature: 44° C.
Nozzle size: 1.2 mm
Nozzle pressuer: 3.0 bar

After coating, the co-granules were dried for 10 min. to reach a temperature of 75° C. and finally cooled.

### Co-Granule 2:

5 kg Na₂SO₄ cores sieved to 180-250 microns is loaded into a Niro MP-1 top-spray fluid bed. The following mixture is coated onto the cores:
450 g phytase liquid concentrate
ca. 2000 g liquid carbohydrase
50 g Dextrin, Avedex W80

| | |
|---|---|
| 3300 g | Water |

### Co-Granule 3:

3.0 kg of co-granule 2 is loaded into a Niro MP-1 top-spray fluid bed.

The following mixture was coated onto the cores:

| | | |
|---|---|---|
| 1200 | g | Na₂SO₄ |
| 50 | g | Dextrin, Avedex W80 |
| 3200 | g | Water |

### Co-Granule 4:

3.0 kg of co-granule 2 is loaded into a MP-1 top spray fluid bed.

The following mixture was coated onto the cores:

| | | |
|---|---|---|
| 1200 | g | MgSO₄•7H₂O |
| 50 | g | Dextrin, Avedex W80 |
| 2000 | g | Water |

The following bed settings are used during coating of co-granule 1, 2, 3 and 4:
Air flow: 175 kg/h
Inlet air temperature: 80° C.
Product temperature: 42-46°
1.2 mm nozzle
3.6 bar nozzle pressure
After coating the co-granules are dried to a product temperature of 60° C.

The method used in Example 1 was applied to prepare products 1-9, wherein the enzymes used in Example 1 were substituted as required for the desired products, for application as described for co-granule 1 or co-granule 2. Products 10-21 are prepared by the method used in Example 1 for application as described for co-granule 1 or co-granule 2.

### Example 2—Combining a Phytase and a Xylanase

The phytase from the product SEQ ID NO:18 (200 ppm) and the xylanase having SEQ ID NO:1 were combined as in the co-granule described according to Example 1.

| Product 1 (unit/g product) | Product 2 (unit/g product) |
|---|---|
| 12000 FYT/g of phytase | 10000 FYT/g of phytase |
| 800 FXU-W/g of xylanase | 360 FXU-W/g of xylanase |

A composition comprising multiple co-granules of either product 1 or product 2 were prepared with high homogeneity of the enzymes so as to give the same protein load pr. g in the enzyme product, while having a better activity distribution in the feed when applied, compared to the single solutions.

### Example 3—Combining an Amylase and a Protease (HSP)

The amylase having SEQ ID NO: 2 (89 ppm) and the protease having SEQ ID NO:16 (50 ppm) were combined as in the co-granule described according to Example 1.

### Product 3 (Unit/q Product)

641 KNU—Kilo Novo Units amylase
240.000 NFP protease

A composition comprising multiple co-granules of product 1 were prepared with high homogeneity of the enzymes so as to give the same protein load pr. g in the enzyme product, while having a better activity distribution in the feed when applied, compared to the single solutions.

### Example 4—Combining Carbohydrases and Protease (MVP)

The combination of endo 1,4-beta-xylanase, endo-1,3(4)-beta-glucanase and endo 1,4-beta-glucanase having SEQ ID NO:3, SEQ ID NO:4, and SEQ ID NO: 5 (100 ppm), respectively, and the beta-glucanase (250 ppm) having SEQ ID NO:8, and the protease having SEQ ID NO:15 (200 ppm), were combined as in the co-granule described according to Example 1.

### Product 4 (Unit/q Product)

1.080 units Endo 1,4-Beta Xylanase
320 units Endo 1,3(4)-Beta-Glucanase
320 units Endo 1,4 B-Glucanase
50 FBG—Fungal B-Glucanase Units
60.000 PROT-Keratinase Units

A composition comprising multiple co-granules of product 1 were prepared with high homogeneity of the enzymes so as to give the same protein load pr. g in the enzyme product, while having a better activity distribution in the feed when applied, compared to the single solutions.

### Example 5—Combining Carbohydrases and Phytase

The combination of endo 1,4-beta-xylanase, endo-1,3(4)-beta-glucanase and endo 1,4-beta-glucanase having SEQ ID NO:3, SEQ ID NO:4, and SEQ ID NO: 5 (50 ppm), respectively, and the phytase having SEQ ID NO: 18 (75 ppm) were combined as in the co-granule described according to Example 1.

### Product 5 (Unit/q Product)

900 units endo 1,4-beta xylanase
267 units endo 1,3(4)-beta-glucanase
233 units endo 1,4-beta-glucanase
10000 FYT/g of phytase

A composition comprising multiple co-granules of product 1 were prepared with high homogeneity of the enzymes so as to give the same protein load pr. g in the enzyme product, while having a better activity distribution in the feed when applied, compared to the single solutions.

### Example 6—Combining Carbohydrases and Phytase

The combination of endo 1,4-beta-xylanase, endo-1,3(4)-beta-glucanase and endo 1,4-beta-glucanase having SEQ ID NO:3, SEQ ID NO:4, and SEQ ID NO: 5(50 ppm), respectively, and the phytase having SEQ ID NO:19 (37.5 ppm) were combined as in the co-granule described according to Example.

### Product 6 (Unit/q Product)

900 units Endo 1,4-Beta Xylanase
267 units Endo 1,3(4) B-Glucanase
233 units Endo 1,4 B-Glucanase
7500 FYT/g of phytase

A composition comprising multiple co-granules of product 1 were prepared with high homogeneity of the enzymes so as to give the same protein load pr. g in the enzyme product, while having a better activity distribution in the feed when applied, compared to the single solutions.

### Example 7—Combining Carbohydrases and Phytase

The phytase having SEQ ID NO:19 (37.5 ppm) and the xylanase having SEQ ID NO:1 (20 ppm), were combined as in the co-granule described according to Example 1.

### Product 7 (Unit/q Product)

7500 FYT/g of phytase
500 FXU-W/g of xylanase

A composition comprising multiple co-granules of product 1 were prepared with high homogeneity of the enzymes so as to give the same protein load pr. g in the enzyme product, while having a better activity distribution in the feed when applied, compared to the single solutions.

### Example 8—Combining Carbohydrases

The combination of endo 1,4-beta-xylanase, endo-1,3(4)-beta-glucanase and endo 1,4-beta-glucanase having SEQ ID NO:3, SEQ ID NO:4, and SEQ ID NO: 5 (100 ppm), respectively, and the xylanase having SEQ ID NO:1 (30 ppm), and beta-glucanase (200 ppm) having SEQ ID NO:8 and the amylase having SEQ ID NO:2 (70 ppm) were combined as in the co-granule described according to Example 1.

### Product 8 (Unit/q Product)

1350 units Endo 1,4-Beta Xylanase
400 unitsEndo 1,3(4) B-Glucanase
350 units Endo 1,4 B-Glucanase
750 FXU—Fungal Xylanase Units
50 FBG—Fungal B-Glucanase Units
315 KNU—Kilo Novo Units Amylase

A composition comprising multiple co-granules of product 1 were prepared with high homogeneity of the enzymes so as to give the same protein load pr. g in the enzyme product, while having a better activity distribution in the feed when applied, compared to the single solutions.

### Example 9—Combining Carbohydrases and Protease (HSVP)

The beta-glucanase (200 ppm) having SEQ ID NO:8 and the amylase having SEQ ID NO:2 (67 ppm) and the protease having SEQ ID NO:16 (50 ppm) were combined as in the co-granule described according to Example 1.

### Product 9

FBG—Fungal Beta-Glucanase Units
KNU—Kilo Novo Units Amylase
NFP

A composition comprising multiple co-granules of product 1 were prepared with high homogeneity of the enzymes so as to give the same protein load pr. g in the enzyme product, while having a better activity distribution in the feed when applied, compared to the single solutions.

### Example 10—Products 10 to 21

The co-granules prepared according to Example, are modified to comprise the enzyme content of any one of Products 10, 11, 12, 13, 14, 15, 16, 17, 18, 19, 20 and 21 prepared with high homogeneity of the enzymes so as to give the same protein load pr. g in the enzyme product, while having a better activity distribution in the feed when applied, compared to the single solutions.

### Product 10: Co-Granulate Comprising a Xylanase, Amylase and Protease

10i) Xylanase: SEQ ID NO: 49 (available from DuPont®),
10ii) Amylase: SEQ ID NO:50 (available from DuPont®);
10iii) Protease: SEQ ID NO:51 (available from DuPont® under the tradename Axtra® Pro).

### Product 11: Co-Granulate Comprising a Xylanase, an Amylase, a Protease and a Phytase

11i) Xylanase: SEQ ID NO: 49 (available from DuPont®),
11ii) Amylase: SEQ ID NO:50 (available from DuPont®);
11iii) Protease: SEQ ID NO:51 (available from DuPont under the tradename Axtra® Pro); and
11iv) Phytase: SEQ ID NO:52 (available from Dupont under the tradename Axtra® Phy Gold).

### Product 12: A Co-Granulate Comprising a Xylanase and a Phytase

12i) Xylanase: SEQ ID NO: 49 (available from DuPont®), and
12ii) Phytase: SEQ ID NO:52 (available from DuPont under the tradename Axtra® Phy Gold).

### Product 13: A Co-Granulate Comprising a Phytase and a Carbohydrase

13i) Phytase: SEQ ID NO:53 (available from BASF under the tradename NatuPhos® E);
13ii) Carbohydrase: SEQ ID NO: 54 (available from BASF under the tradename Natugrain® TS)

### Product 14: A Co-Granulate Comprising a Phytase and a Carbohydrase

14i) Phytase. polypeptide originating from Buttiauxella sp and available from Adisseo under the tradename Rovabio® PhyPlus or Rovabio® Advance PHY;
14ii) Carbohydrase: polypeptide originating from Penicillium funiculosum available from Adisseo under the tradename Rovabio® Excel or Rovabio® Advance.

### Product 15: A Co-Granulate Comprising a Phytase and a Carbohydrase

15i) Phytase: SEQ ID NO:57:
15ii) Carbohydrase: SEQ ID NO:58:

### Product 16: A Co-Granulate Comprising a Phytase and a Mannanase

16i) Phytase: SEQ ID NO:53 available from BASF® under the tradename NatuPhos® E;
16ii) Mannanase: polypeptide available from BASF® under the tradename Natupulse®.

### Product 17: A Co-Granulate Comprising a Phytase and a Mannanase

17i) Mannanase: polypeptide available from BASF® under the tradename Natupulse®;
17ii) Carbohydrase: SEQ ID NO: 54 available from BASF® under the tradename Natugrain® TS.

### Product 18: A Co-Granulate Comprising a Phytase and One or Two Carbohydrases

18i) Phytase: SEQ ID NO: 55 available from Huvepharma® under the tradename Optiphos® Plus);
18ii) Carbohydrase: SEQ ID NO:56 available from Huvepharma® under the tradename Hostazym®
18iii) Carbohydrase: available from Huvepharma® under the tradename Huvezym® neXo.

### Product 19: A Co-Granulate Comprising a Phytase, a Xylanase, a Protease and a Mannanase

19i)Phytase: SEQ ID NO:59 available from VTR® Biotech under the tradename Microtech®;
19ii) Xylanase: SEQ ID NO:60 available from VTR Biotech available from VTR® Biotech under the tradename Yiduozyme®
19iii) Protease: SEQ ID NO:61 available from VTR® Biotech
19iv) Mannanase: SEQ ID NO:62 available from VTR® Biotech

### Product 20: Co-Granulate Comprising a Phytase, a Xylanase, and a Protease

20i)Phytase: SEQ ID NO:59 available from VTR® Biotech under the tradename Microtech®;
20ii) Xylanase: SEQ ID NO:60 available from VTR® Biotech under the tradename Yiduozyme®
20iii) Protease: SEQ ID NO:61 available from VTR® Biotech

### Product 21: A Co-Granulate Comprising a Combination of a Phytase and a Protease

21i) Phytase: SEQ ID NO:59 (available from VTR® Biotech under the tradename Microtech®
21ii) Protease: SEQ ID NO:61 available from VTR® Biotech

1. A granulate composition comprising a plurality of co-granules, said co-granules comprising a core unit and a coating, wherein the core comprises at least two classes of enzymes, wherein the granulate composition has a homogeneity such that i) the quantity of each enzyme in a co-granule is 80-120% of the quantity of the same enzyme in at least 90% of the co-granules in said composition; and/or ii) wherein the ratio of the at least two classes of enzymes within a co-granule is 80% to 120% the ratio of the same two enzymes within at least 90% of the co-granules in said composition.

2. The granulate composition according to claim 1, wherein the at least two classes of enzymes are selected from carbohydrases, phytases, muramidases, and protease.

3. The granulate composition according to claim 1, wherein the at least two classes of enzymes are selected from xylanases, amylases, mannanases, cellulases, polygalacturonases, beta-glucanases, phytases, protease, and muramidase.

4. The granulate composition according to claim 1, said at least two classes of enzymes comprising a combination selected from the group consisting of
a. a combination of a phytase and a xylanase;
b. a combination of an amylase and a protease;
c. a combination of a xylanase and a protease;
d. a combination of a phytase, a protease and a xylanase;
e. a combination of an endo 1,4-beta-xylanase, an endo-1,3(4)-beta-glucanase, a endo-1,4-beta-glucanase, a beta-glucanase and a protease;
f. a combination of a 1,4-beta-xylanase, an endo-1,4-beta-glucanase and a phytase;
g. a combination of a 1,4-beta-xylanase, an endo-1,3(4)-beta-glucanase, an endo 1,4-beta-glucanase and a phytase;
h. a combination of an endo 1,4-beta-xylanase and an amylase;
i. a combination of an endo 1,4-beta-xylanase, a endo 1,4-beta-glucanase and an amylase;
j. a combination of an endo 1,4-beta-xylanase, endo-1,3(4)-beta-glucanase and endo 1,4-beta-glucanase with the xylanase beta-glucanase (200 ppm) and with the amylase;
k. a combination of a beta-glucanase, an amylase and a protease.
l. a combination of a xylanase, an amylase and a protease;
m. a combination of a xylanase, an amylase, a protease and a phytase;
n. a combination of a protease and a phytase;
o. a combination of a phytase and a carbohydrase;
p. a combination of a phytase and a mannanase;
q. a combination of a mannanase and a carbohydrase; and
r. a combination of a phytase, xylanase, protease and mannanase.

5. The granulate composition according to claim 2, said at least two classes of enzymes comprising at least one enzyme selected from:
a. polypeptides having at least 90% sequence identity to SEQ ID NO: 18, SEQ ID NO: 19, SEQ ID NO:41, SEQ ID NO:42, SEQ ID NO:43, SEQ ID NO:44, SEQ ID NO:45, SEQ ID NO:48, SEQ ID NO: 52, SEQ ID NO: 53, SEQ ID NO: 55, SEQ ID NO: 57, and SEQ ID NO: 58;
b. polypeptides having at least 90% sequence identity to SEQ ID NO:1, SEQ ID NO:3, SEQ ID NO:21, SEQ ID NO:22, SEQ ID NO:23, SEQ ID NO:25, SEQ ID NO:26, SEQ ID NO:33, SEQ ID NO:35, SEQ ID NO:38, SEQ ID NO: 49 and SEQ ID NO:60;
c. SEQ ID NO: 15, SEQ ID NO: 16, SEQ ID NO: 32, SEQ ID NO: 37, SEQ ID NO: 39, SEQ ID NO: 40, SEQ ID NO: 51 and SEQ ID NO: 61;
d. SEQ ID NO: 2, SEQ ID NO: 36 and SEQ ID NO:50;
e. SEQ ID NO: 5, SEQ ID NO: 6, SEQ ID NO: 11, SEQ ID NO: 12, SEQ ID NO: 13, SEQ ID NO: 14, SEQ ID NO: 30, SEQ ID NO: 54, SEQ ID NO: 56 and SEQ ID NO: 58;
f. SEQ ID NO: 17, SEQ ID NO: 24 and SEQ ID NO:62.

6. The granulate composition according to claim 1, said at least two classes of enzymes comprising a polypeptide having at least 80% sequence identity to SEQ ID NO: 15, SEQ ID NO: 16, SEQ ID NO: 32, SEQ ID NO: 39, and/or SEQ ID NO: 40

7. The granulate composition according to claim 1, said at least two classes of enzymes comprising a polypeptide having at least 80% sequence identity to SEQ ID NO: 2, SEQ ID NO: 34 and/or SEQ ID NO:36.

8. The granulate composition according to claim 1, said at least two classes of enzymes comprising a polypeptide having at least 80% sequence identity to SEQ ID NO: 1, SEQ ID NO:3, SEQ ID NO:21, SEQ ID NO:22, SEQ ID NO:23, SEQ ID NO:25, SEQ ID NO:26, SEQ ID NO:33, and/or SEQ ID NO:35.

9. The granulate composition according to claim 1, said at least two classes of enzymes comprising a polypeptide having at least 800 sequence identity to SEQ ID NO:18, SEQ ID NO:19, SEQ ID NO:41, SEQ ID NO:42, SEQ ID NO:43, SEQ ID NO:44, SEQ ID NO:45 and/or SEQ ID NO:48.

10. The granulate composition according to claim 1, wherein i) the quantity of each enzyme in a co-granule is 90-110% of the quantity of the same enzyme in at least 90% of co-granules in said composition; and/or ii) wherein the ratio of the at least two classes of enzymes within a co-granule is 90% to 110% the ratio of the same two enzymes within at least 90% of co-granules in said composition.

11. The granulate composition according to claim 1, wherein i) the quantity of each enzyme in a co-granule is 80-120% of the quantity of the same enzyme in at least 95% of co-granules in said composition; and/or ii) wherein the ratio of the at least two classes of enzymes within a co-granule is 80% to 120% the ratio of the same two enzymes within at least 95% of co-granules in said composition.

12. The granulate composition according to claim 1, wherein i) the quantity of each enzyme in a co-granule is 90-110% of the quantity of the same enzyme in at least 95% of co-granules in said composition; and/or ii) wherein the ratio of the at least two classes of enzymes within a co-granule is 90% to 110% the ratio of the same two enzymes within at least 95% of co-granules in said composition.

13. The granulate composition according to claim 1, wherein the at least two classes of enzymes are present in a ratio of about 5:1 (wt:wt).

14. A co-granule comprising a core and a coating, wherein the core comprises at least two enzyme classes.

15. An animal feed comprising an animal feed additive, said additive comprising co-granules, each co-granule comprising at least two classes of enzymes, said co-granules comprising a core unit and a protective coating.

16. A method of manufacturing a co-granule comprising combining two enzyme classes onto a core and coating the enzyme-containing core with a coating.


SEQUENCE LISTING (62 sequences)

SEQ ID NO: 1 - protein, 225 aa, Thermomyces lanuginosus
MVGFTPVALAALAATGALAFPAGNATELEKRQTTPNSEGWHDGYYYSWWSDGGAQATYTNLEGGTYEISWGDGGNLVGGKGWNPGLNARAIHFEGVYQPNGNSYLAVYGWTRNPLVEYYIVENFGTYDPSSGATDLGTVECDGSIYRLGKTTRVNAPSIDGTQTFDQYWSVRQDKRTSGTVQTGCHFDAWARAGLNVNGDHYYQIVATEGYFSSGYARITVADVG

SEQ ID NO: 2 - protein, 486 aa, Bacillus amyloliquefaciens
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGKAWDWEVDTEFGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVS

SEQ ID NO: 3 - protein, 223 aa, Trichoderma reesei
MVSFTSLLAGVAAISGVLAAPAAEVESVAVEKRQTIQPGTGYNNGYFYSYWNDGHGGVTYTNGPGGQFSVNWSNSGNFVGGKGWQPGTKNKVINFSGSYNPNGNSYLSVYGWSRNPLIEYYIVENFGTYNPSTGATKLGEVTSDGSVYDIYRTQRVNQPSIIGTATFYQYWSVRRNHRSSGSVNTANHFNAWAQQGLTLGTMDYQIVAVEGYFSSGSASITVS

SEQ ID NO: 4 - protein, 459 aa, Trichoderma reesei
MAPSVTLPLTTAILAIARLVAAQQPGTSTPEVHPKLTTYKCTKSGGCVAQDTSVVLDWNYRWMHDANYNSCTVNGGVNTTLCPDEATCGKNCFIEGVDYAASGVTTSGSSLTMNQYMPSSSGGYSSVSPRLYLLDSDGEYVMLKLNGQELSFDVDLSALPCGENGSLYLSQMDENGGANQYNTAGANYGSGYCDAQCPVQTWRNGTLNTSHQGFCCNEMDILEGNSRANALTPHSCTATACDSAGCGFNPYGSGYKSYYGPGDTVDTSKTFTIITQFNTDNGSPSGNLVSITRKYQQNGVDIPSAQPGGDTISSCPSASAYGGLATMGKALSSGMVLVFSIWNDNSQYMNWLDSGNAGPCSSTEGNPSNILANNPNTHVVFSNIRWGDIGSTTNSTAPPPPPASSTTFSTTRRSSTTSSSPSCTQTHWGQCGGIGYSGCKTCTSGTTCQYSNDYYSQCL

SEQ ID NO: 5 - protein, 514 aa, Trichoderma reesei
MYRKLAVISAFLATARAQSACTLQSETHPPLTWQKCSSGGTCTQQTGSVVIDANWRWTHATNSSTNCYDGNTWSSTLCPDNETCAKNCCLDGAAYASTYGVTTSGNSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEPSSNNANTGIGGHGSCCSEMDIWEANSISEALTPHPCTTVGQEICEGDGCGGTYSDNRYGGTCDPDGCDWNPYRLGNTSFYGPGSSFTLDTTKKLTVVTQFETSGAINRYYVQNGVTFQQPNAELGSYSGNELNDDYCTAEEAEFGGSSFSDKGGLTQFKKATSGGMVLVMSLWDDYYANMLWLDSTYPTNETSSTPGAVRGSCSTSSGVPAQVESQSPNAKVTFSNIKFGPIGSTGNPSGGNPPGGNPPGTTTTRRPATTTGSSPGPTQSHYGQCGGIGYSGPTVCASGTTCQVLNPYYSQCL

SEQ ID NO: 6 - protein, 471 aa, Trichoderma reesei
MIVGILTTLATLATLAASVPLEERQACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDSHCALPDALQPAPQAGAWFQAYFVQLLTNANPSFL

SEQ ID NO: 7 - protein, 797 aa, Trichoderma reesei
MVNNAALLAALSALLPTALAQNNQTYANYSAQGQPDLYPETLATLTLSFPDCEHGPLKNNLVCDSSAGYVERAQALISLFTLEELILNTQNSGPGVPRLGLPNYQVWNEALHGLDRANFATKGGQFEWATSFPMPILTTAALNRTLIHQIADIISTQARAFSNSGRYGLDVYAPNVNGFRSPLWGRGQETPGEDAFFLSSAYTYEYITGIQGGVDPEHLKVAATVKHFAGYDLENWNNQSRLGFDAIITQQDLSEYYTPQFLAAARYAKSRSLMCAYNSVNGVPSCANSFFLQTLLRESWGFPEWGYVSSDCDAVYNVFNPHDYASNQSSAAASSLRAGTDIDCGQTYPWHLNESFVAGEVSRGEIERSVTRLYANLVRLGYFDKKNQYRSLGWKDVVKTDAWNISYEAAVEGIVLLKNDGTLPLSKKVRSIALIGPWANATTQMQGNYYGPAPYLISPLEAAKKAGYHVNFELGTEIAGNSTTGFAKAIAAAKKSDAIIYLGGIDNTIEQEGADRTDIAWPGNQLDLIKQLSEVGKPLVVLQMGGGQVDSSSLKSNKKVNSLVWGGYPGQSGGVALFDILSGKRAPAGRLVTTQYPAEYVHQFPQNDMNLRPDGKSNPGQTYIWYTGKPVYEFGSGLFYTTFKETLASHPKSLKFNTSSILSAPHPGYTYSEQIPVFTFEANIKNSGKTESPYTAMLFVRTSNAGPAPYPNKWLVGFDRLADIKPGHSSKLSIPIPVSALARVDSHGNRIVYPGKYELALNTDESVKLEFELVGEEVTIENWPLEEQQIKDATPDA

SEQ ID NO: 8 - protein, 436 aa, Aspergillus aculeatus
MRTSHLLSQALSLLALGATVEGFNRDRNQACGPHKPFQPLPTSKSRTKTCHVRTHGDGSDDSAYILSAIKQCNNGGKVVFDADKEYIIGTALDLTFLKHIDLDIEGTIQFTNDTDYWQANAFQQVFQNATTFFQLGGEDVNVYGGGTLDGNGQVWYDLYAEDALILRPILFGIIGLNGGTIGPLNLRYSPQWYNLIANSTNVLYDGINISGYSKNPSVTAKNTDGWDTYRSSNIVIQNSVINNGDDCVSFKPNSTDILVQNLHCNGSHGISVGSLGQYLGEVDIVQDILVYNISMYNASDMARIKVWPGVSSALSEDLQGGGGSGRVQNVTYDTAYIDNVDWAIEVTQCYGQSNLTLCNEYPSSLTISDVWFKNFRGKTSGKENPYVGTLVCSSPEVCSNIYTDNINVVSPKGTNDFVCNNVDTSDLSVNCTATSS

SEQ ID NO: 9 - protein, 377 aa, Aspergillus aculeatus
MKLSHMLLSLASLALATALPRTPNHNAATTAFPSTSGLHFTIDGKTGYFAGTNSYWIGFLTNNDDVDLVMSQLAASDLKILRVWGFNDVNTKPTDGTVWYQLHANGTSTINTGADGLQRLDYVVTSAEKYGVKLIINFVNEWTDYGGMQAYVTAYGAAAQTDFYTNTAIQAAYKNYIKAVVSRYSSSAAIFAWELANEPRCQGCDTSVLYNWISDTSKYIKSLDSKHLVTIGDEGFGLDVDSDGSYPYTYGEGLNFTKNLGISTIDFGTLHLYPDSWGTSYDWGNGWITAHAAACKAVGKPCLLEEYGVTSNHCAVESPWQQTAGNATGISADLYWQYGTTFSWGQSPNDGNTFYYNTSDFTCLVTDHVAAINAQSK

SEQ ID NO: 10 - protein, 396 aa, Aspergillus aculeatus
MLLYRGLLLLLHVLLVHSAALQPRLSTATRETCQTSLKCPEGTIIVSNSSHPAAQFKSIQAAFASLPNDNSSHTILVLAGTYHEQLNLTRPGPVTLLGQQPDRSALTDPTRNRVTITWAAANQDNTGQSVDNVYSSVLIVSPTLDASLTGSGTTGFAVPADTPFGNTDFRVYNIDFRNLWAEYSDGPAHAISFSRANGGFYYSGFYSYQDTVYIGKLGNAYFHKSVIAGQTDFLYGFGTAWIQSSSILLRSCGGGITAWKGTNTTFENNYGVYIVDSTVNAANSSLVSEIKGKCALGRPWNSLHRSIFVNSYEDGSIEPSGYINWEDRWAANQTLMAEYRAYGPGFNATGRKEGGVAVLLNAQEYKRYDSLEKVFLTPEGEAGNIAWIDWDIAGKD

SEQ ID NO: 11 - protein, 446 aa, Aspergillus aculeatus
MKLVSIAALLLGAVPIAVNAAAVGGTPNGAKIIPVSDAAGLRSVGAHSSKYPNRKTVTIRPSKNDYDDISSDFLWGIKQANHGGRLLLEKGKKYVIGKKLDLTFLDNIEVQLEGELKFTNNITYWQANNYYYDFQKSITFWRWGGNDIKIFGSGTLNGNGQAWYNAFAGLEILDSSNTFYRPILFLTDNATRVSVEGITQLNSPCWTNFFVNTKDISFDDVYIHAFSTNASALPKNTDGFDSYNVDGLTVTNTRVDIGDDCFSPKPNTTNIFVQNLWCNNTHGVSMGSIGQYPGELDIIENAWIENVTLLNGQNGARLKAWAGPDVGYGRINNITYKNIQIQNTDNPVVLDQCYFDINATECAQYPSSVNITNILFENIWGTSSGKEGLVVADLTCSPAAVCTNVTLQDVSLTSPAGSPAEIICSGITGGIGVDCVDETNSTSTKR

SEQ ID NO: 12 - protein, 440 aa, Aspergillus aculeatus
MRGLFLLALGAIPALVSGQLSGSVGPLTSASTKGATKTCNILSYGAVADNSTDVGPAITSAWAACKSGGLVYIPSGNYALNTWVTLTGGSATAIQLDGIIYRTGTASGNMIAVTDTTDFELFSSTSKGAVQGFGYVYHAEGTYGARILRLTDVTHFSVHDIILVDAPAFHFTMDTCSDGEVYNMAIRGGNEGGLDGIDVWGSNIWVHDVEVTNKDECVTVKSPANNILVESIYCNWSGGCAMGSLGADTDVTDIVYRNVYTWSSNQMYMIKSNGGSGTVSNVLLENFIGHGNAYSLDIDGYWSSMTAVDGDGVQLNNITVKNWKGTEANGATRPPIRVVCSDTAPCTDLTLEDIAIWTESGSSELYLCRSAYGSGYCLKDSSSHTSYTTTSTVTAAPSGYSATTMAADLTTAFGLTASIPIPTIPTSFYPGLTPYSALAG

SEQ ID NO: 13 - protein, 379 aa, Aspergillus aculeatus
MALTTILSLLAVAAQLATAVSVSGAAEGFAKGVTGGGSATAVYPTTTDELVSYLGDSSARVIVLQQTFDFTGTEGTTTATGCAPWGTASACQVAINQNDWCTNYEPDAPSVSVTYDNAGVLGITVASDKTLIGVGSTGIIKGKGLRIVSGASNIIIQNVAITDINEKYVWGGDAITLDDADMVWIDHVTTARIGRQHVVLGTSADNRVTISNSYFDGVTSYSATCDGYAYWGLYFDGSSDLVTLQKNYIYHFSGRSPKVQGNTLLHAVNNYWYDSDGHSFEIGSGGYVLAEGNVFQNIDTPVESPVSGQLFTSPDSTTNAVCSTYLGRACQINGFGSSGTFSQSDTAFLVDFEGKNIATASAYTAIQTTVPSGAGQGNL

SEQ ID NO: 14 - protein, 352 aa, Aspergillus aculeatus
MKPLWIRLWAIFFFGLAVCAAPGVQARVVDGGSIMDQHMRENLCALTFDDGPSANTPQLLDMLSEYGIPATFFLLGKQAERHPDLVRRILAEGHEVGNHSYSHPNLRMLSPEKKLQEIGQTDAILRSLGASPSFLRPPYGAYDNHTVSVAESLGLGIMLWSLDSRDWKSLPANYATLRSTRGTVYDTGALRGIFLFHDTHRRTVEDLPRIIRDLRAGGCQRFVTVSDYLDGLLDPEPGILMTRRPTDSAQPAVADKKPGRLKEQFQEMPPESYPAGTAALPLARTSRPWRSGSGAAETGRPPAAEVDRQAAGENSGPGHDSSVQPTTGAPEAGLPLGSSVFTPGAAAGSADA

SEQ ID NO: 15 - protein, 185 aa, Nocardiopsis prasina
IGGLAYTMGGRCSVGFAATNAAGQPGFVTAGHCGRVGTQVTIGNGRGVFEQSVFPGNDAAFVRGTSNFTLTNLVSRYNTGGYATVAGHNQAPIGSSVCRSGSTTGWHCGTIQARGQSVSYPEGTVTNMTRTTVCAEPGDSGGSYISGTQAQGVTSGGSGNCRTGGTTFYQEVTPMVNSWGVRLRT

SEQ ID NO: 16 - protein, 311 aa, Bacillus sp.
AVPSTQTPWGIKSIYNDQSITKTTGGKGIKVAVLDTGVYTSHLDLAGSAEQCKDFTQSNPLVDGSCTDRQGHGTHVAGTVLAHGGSNGQGVYGVAPQAKLWAYKVLGDKGEGYSDDIAAAIRHVADEASRTGSKVVINMSLGSSAKDSLIASAVDYAYGKGVLIVAAAGNEGPKPNTIGYPAGFVNAVAVAALENVQEKGTYRVADFSSRGNPATAGDYIIQERDIEVSAPGASVESTWYTGGYNTISGTSMATPHVAGLAAKIWSANTSLSHSQLRTELQNRAKVYDIKGGIGAGPGDDYASGFGYPRVK

SEQ ID NO: 17 - protein, 431 aa, Talaromyces Leycettanus
MKLSTLNFLSLAGLVSAQVANYGQCGGQNYSGPTTCNPGWSCQYLNPYYSQCLPATQTTTLTTSTKPTSTSTTTRTSTSTTSTQGGSSSTSIPSKNGLKFTIDGKTAYYAGTNTYWLPFLTNNADVDLVMSHLQQSGLKILRVWGFNDVNTQPGSGTVWFQLLQNGQATINTGANGLQRLDYVVQSAEAHDIKLIINFVNNWNDYGGINAYVNNYGGNATTWYTNSAAQAAYRNYIKAVISRYIGSPAIFAWELANEPRCHGCDTSVIYNWVSSTSAYIKSLEPNRMVCIGDEGMGLTTGSDGSYPFQYTEGTDFEKNLAIPTIDFGTLHLYPSSWGEQDSWGSTWISAHGQACVNAGKPCLLEEYGSTNHCSSEAPWQSTALSTNGIAADSFWQYGDTLSTGQSPNDGYTIYYGSSDYTCLVTNHISQFQ

SEQ ID NO: 18 - protein, 433 aa, Citrobacter braakii
MSTFIIRLLFFSLLCGSFSIHAEEQNGMKLERVVIVSRHGVRAPTKFTPIMKNVTPDQWPQWDVPLGWLTPRGGELVSELGQYQRLWFTSKGLLNNQTCPSPGQVAVIADTDQRTRKTGEAFLAGLAPKCQIQVHYQKDEEKNDPLFNPVKMGKCSFNTLQVKNAILERAGGNIELYTQRYQSSFRTLENVLNFSQSETCKTTEKSTKCTLPEALPSELKVTPDNVSLPGAWSLSSTLTEIFLLQEAQGMPQVAWGRITGEKEWRDLLSLHNAQFDLLQRTPEVARSRATPLLDMIDTALLTNGTTENRYGIKLPVSLLFIAGHDTNLANLSGALDLNWSLPGQPDNTPPGGELVFEKWKRTSDNTDWVQVSFVYQTLRDMRDIQPLSLEKPAGKVDLKLIACEEKNSQGMCSLKSFSRLIKEIRVPECAVTE

SEQ ID NO: 19 - protein, 411 aa, Citrobacter braakii
EEQNGMKLERVVIVSRHGVRAPTKFTPIMKCVTPDQWPQWDVPLGWLTPRGCELVSYLGQYQRLWFTSKGLLPNQTCPSPGQVAVIADTDQRTRKTGECFLAGLAPKCQIQVHYQKDEEKPDPLFNPVKMGKCQFNTLQVCNAILERAGGNIELFTQRYQSSFRTLENVLNFSQSECCKTTEKSTKCTLPEALPSELKCTPDLVSLTGAWSLSSTLTEIFLLQEAQGMPQVAWGRITGEKEWRDLLSLHNAQFDLLQRTPEVARSRATPLLDLIDTALLTNGTTENRYGIKLPVSLLFIAGHDTNLANLSGALDLNWSLPGQPDNTPPGGELVFEKWKRTSDNTDWVQVSFVYQTLRDMRDIQPLSLEKPAGKVDLKLIACEEKNSQGMCSLKSFSRLIKEIRVPECAVTE

SEQ ID NO: 20 - protein, 681 aa, Acremonium alkalophilum
METLYSLEULEUPROSERLEUILEGLYLEUALASERLEUALASERLEUALAVALALAARGILEPROGLYPHEASPILESERGLYTRPGLNPROTHRTHRASPPHEALAARGALATYRALAASNGLYASPARGPHEVALTYRILELYSALATHRGLUGLYTHRTHRPHELYSSERSERALAPHESERARGGLNTYRTHRGLYALATHRGLNASNGLYPHEILEARGGLYALATYRHISPHEALAGLNPROALAALASERSERGLYALAALAGLNALAARGTYRPHEALASERASNGLYGLYGLYTRPSERLYSASPGLYILETHRLEUPROGLYALALEUASPILEGLUTYRASNPROASNGLYALATHRCYSTYRGLYLEUSERGLNSERALAMETVALASNTRPILEGLUASPPHEVALTHRTHRTYRHISGLYILETHRSERARGTRPPROVALILETYRTHRTHRTHRASPTRPTRPTHRGLNCYSTHRGLYASNSERASNARGPHEALAASNARGCYSPROLEUTRPILEALAARGTYRALASERSERVALGLYTHRLEUPROASNGLYTRPGLYPHETYRTHRPHETRPGLNTYRASNASPLYSTYRPROGLNGLYGLYASPSERASNTRPPHEASNGLYASPALASERARGLEUARGALALEUALAASNGLYASP

SEQ ID NO: 21 - protein, 213 aa, Bacillus subtilis
MFKFKKNFLVGLTAAFMSISMFSATASAAGTDYWQNWTDGGGTVNAVNGSGGNYSVNWSNTGNFVVGKGWTTGSPFRTINYNAGVWAPNGNGYLTLYGWTRSPLIEYYVVDSWGTYRPTGTYKGTVKSDGGTYDIYTTTRYNAPSIDGDNTTFTQYWSVRQSKRPTGSNAAITFSNHVNAWKSHGMNLGSNWAYQVLATEGYKSSGSSNVTVW

SEQ ID NO: 22 - protein, 191 aa, Trichoderma reesei
QCIQPGTGYNNGYFYSYWNDGHGGVTYCNGPGGQFSVNWSNSGNFVGGKGWQPGTKNRVINFSGSYNPNGNSYLSVYGWSRNPLIEYYIVENFGTYNPSTGATKLGEVTSDGSVYDIYRTQRVNQPSIIGTATFYQYWSVRRNHRSSGSVNTANHFNAWAQQGLTLGTMDYQIVAVEGYFSSGSASITVSD

SEQ ID NO: 23 - protein, 344 aa, Thermopolyspora flexuosa
MNEPLTITQARRRRRLGLRRIVTSAFALALAIAGALLPGTAHADTTITQNQTGYDNGYFYSFWTDAPGTVSMTLHSGGSYSTSWRNTGNFVAGKGWSTGGRRTVTYNASFNPSGNAYLTLYGWTRNPLVEYYIVESWGTYRPTGTYKGTVTTDGGTYDIYETWRYNAPSIEGTRTFQQFWSVRQQKRTSGTITIGNHFDAWARAGMNLGSHDYQIMATEGYQSSGSSTVSISEGGNPGNPGNPGNPGNPGNPGNPGGGCVATLSAGQQWSDRYNLNVSVSGSNNWTVRMDVPYPARIIATWNIHAQWPESQVLIARPNGNGNNWGVTIQHNGNWTWPTVTCTAN

SEQ ID NO: 24 - protein, 594 aa, Paenibacillus woosongensis
MARKRLKSFATMLLAMAVITASAIVSGGQASAAATPVKGFYVNGTTLYDATGKPFVMRGVNHAHTWFKNDLAAAIPAIAATGSNTVRIVLSDGGQWTRDDVASVQNIIALCDQYNLITMLEVHDATGSDSASTLNRAVDYWISIKDALIGKEDRVIVNIANEWFGSWSSDGWASGYQAAIPALRNAGIKNTLVVDAAGWGQYPNSIFEKGLAVASTDPLKNMIFSIHMYEYAGGDAATVKSNIDNALALNIPVIIGEFGHKHSSGDVDEATILSYTQQKGVGWLAWSWYGNGGGVEYLDLATGPAGSLTDWGKTVVNSTYGTLATSQLSGIYTTPGYQPVPDNGNTPLPTVPAAPTGLTAAAGNGQAVLSWNASSGATSYAVKRAAVSGGPYTTIASNVAATSYTDTGLTNGNTYYYVVSAANSAGTSSHSTQASVTPQGTPVPSSSLVLQYRAGDINSSDNQIKPYFNIKNNGTSPVNLSSLKLRYYFTKDGNQTMNAWIDWAQVGSSNIQTAFGSVSGTNADTYVELSFTAAAGSIAPDGQTGDIQLRMSKADWSNFNENNDYSFDATKTSYTDWNKVTLYNNGDLVWGIEP

SEQ ID NO: 25 - protein, 223 aa, Trichoderma reesei; identical to SEQ ID NO: 3

SEQ ID NO: 26 - protein, 223 aa, Trichoderma viride
MVSFTSLLAGVAPISGVLAAPAAEVESVDVEKRQTIQPGTGYNNGYFYSYWNDGHGGVTYTNGPGGQFSVNWSNSGNFVGGKGWQPGTKNKVINFSGTYNPNGNSYLSVYGWSRNPLIEYYIVENFGTYNPSTGATKLGEVTSDGSVYDIYRTQRVNQPSIEGTSTFYQYWSVRRTHRSSGSVNTANHFNAWASHGLTLGTMDYQIVAVEGYFSSGSASITVS

SEQ ID NO: 27 - protein, 530 aa, Talaromyces funiculosus
MSALNSFSMYKNAIILGSLLATAGAQQIGTYTAETHPSLSWSTCKSGGSCTTNAGSITLDANWRWVHGVNTSTNCYTGNTWNSAICDTDASCAQDCALDGADYSGTYGITTSGNSLRLDFVTGSNVGSRTYLMADNTHYEIFDLLNQEFTFTVDVSKLPCGLNGALYFVTMDADGGVSKYPNNKAGAQYGVGYCDSQCPRDLKFIGGQANVEGWTPSANNSNTGIGTHGSCCAELDIWEANSISEALTPHPCDSATPTICTADDCGGTYSDNRYAGTCDPDGCDFNPYRLGNTNFYGAGMTVDTTKPFTVVTQFVTDDGTSSGSLSEIRRYYVQNGVVIPQPSSKISGISGNVIDSTYCAAEASTFNATTSFTKHGGLAGMGAGLKNGMVLVMSLWDDYSVGMRWLDSTYPTDAAGTPGAARGTCATTSGDPKTVESQDGSAYVVFSDIRVGPFNSTFSGTPGSPGTGTTTTSVGSTSIGSTSTTTSSTSTGTGVASHYGQCGGTGWNGPTSCASGYTCTVVNPYYSQCL

SEQ ID NO: 28 - protein, 462 aa, Talaromyces funiculosus
MLRYLSIVAATAILTGVEAQQSVWGQCGGQSWTGATSCAAGSTCSTINPYYAQCVPGTATSTTLVTTTSPTTSTTSTSVGTTPPPTTSTSKVTTTATTPAASGNPFSGYQLYANPYYSSEVHTLALPSLTGSLAAAATKAAEIPSFVWLDTAAKVPTMGTYLANIQAANKAGASPPIAGIFVVYDLPDRDCAAAASNGEFTVANNGVANYKAYIDSIVKQLKAYPDVHTILIIEPDSLANMVTNLSTAKCSEAQSAYYECVNYALINLNLANVAMYIDAGHAGWLGWPANLSPAAELFATVYKNASSPASLRGLATNVANYNAWSISSPPSYTSGDSNYDEKLYINALSPLLTSNGWPNAHFIMDTSRNGVQPTKQQAWGDWCNVIGTGFGVQPTTNTGDPLEDAFVWVKPGGESDGTSNSSATRYDFHCGYTDALQPAPEAGSWFQAYFVQLLTNANPALV

SEQ ID NO: 29 - protein, 239 aa, Talaromyces funiculosus
MQLTLLLGLAATAAAAALQQSLCSQYSSYSSGPYTVNNNLWGESSGSGSQCTYVNSISSSGVSWSTTWNWQGSSSQVKSYANSQLSGLTKKLVSSLHSIPTSVQWSYSNTNIVADVSYDLFTAADINHVTYSGDYELMIWLGRFGGAQPLGSQIGTANVGGTTWQLWYGVNGSQKTYSFVASGQQTSWNGDILQFFSYLESSHGFPASSQYLIDLQFGTEPFTGSQTTLTVNNWSASVN

SEQ ID NO: 30 - protein, 1093 aa, Talaromyces funiculosus
MLSRALTLSTLVGGVLSIPIEKRACPNIHVFGARETTVSPGYGSSITVVDDVLNSYPGSTAEAINYPACGGQSSCGGISYSSSVAQGIAAVASAVNSFHSECPSSEIILVGYSQGAEIMDVALCGGGDPNQGYTNTAVQLSASAVSMVKAAIFMGDPLFRAGLPYEVGTCAAGGFDERPAGFSCPSASLIKSYCDASDPYCCNGNNAATHQGYGAEYGNQALAFIKSKLSGSTSTGNTTTGTGGGTVAEWGQCGGQGWTGATACKYERQVLGAGTQPQALEAAIAAAENYMRALKLADTPREKQLLDSKCKDWISRAEKIKRDKSWQPSSQVGNRSLKEPISKRKLSTREEIILLESAKLNGFLFPPWRNDPNPQEFELIGNEGQFEDKTLRLSVPQKEVFNGWKRPAELVKEHIRDSVLPDTPNPAETNLVMDLVQDITTDCSVIASLCAAAARSERGHGCLFHNAIFPFGCSKSSPHTSISGKYIFRLYFNGCYRKVVIDDRLPSSKTSRSLFAFDRNAPESFWPALVEKAYLKVRGGYDFPGSNSGTDLWVLTGWIPEQIFLHHEDVAPDQLWKRISSAFQYGDVVLTLGTGELNSREERLQGLIGRHDYAVLDLTESEGYYRLLIKNPWADSSSSTPQSHRHASEQSVLDTNATPLLPGSFWMDYDQIFQNFDNMYLNWNPGLFTTRQDIHFTWDLSKSHFNSTCFANSPQFSISTAVGGPVWLLLSKHFRTRSATNPSAESGFISIYVSRKGGHRIFTSDDAFYRGPFVDSPNTLAKIDMEANSTYTIVIAEQSLPLVSQNFTLSAFTRNEIVLKPAEDKFLHTSRVESEWSPTTAGGNTESPRYPSNPQHVLQLSETADLLILLETPEPDMAVHAKLFWSDGKRISSVKKRDIVTDSGHYRPGCVVAETEQLRPGAYTLVCSTFLPDQIAKFTIWVFSTRKCHVKALPSESAGRLAVLSDVGILPPAVDRILAPLTVSRLTRLKLIARSRNSTIGTRHVTSSPMLLTVELGQGPYKEVLASSENGEYSDAVSGIRVPDFDVRPEDAEKGGIWIVIERIGGPVGQVTDCVDVEALGEERVEIGEWITS

SEQ ID NO: 31 - protein, 389 aa, Talaromyces funiculosus
MLWTVPLLLVIPAVTAQSPVYGQCGGESWTGATTCASGSCCTYSNAWYSQCLPCSGGSGTTTSTTLTTTTKPTSTSTTSTTSKTGSSTSCPLPSQYKWTSSGSLANPMSGYLSLKDFSHVPYNGQHLVYASDVNNGGNYGSMNFALFSDWSQMGSAKQTQMSQGAVAPDLLYFEPKDIWVLGYEWCEWAFCYLTSTNPTNANGWSSPQPLYEGTLSGTGPIDPAIIADDENVYLFFAGDNGNIYRASMPIGNFPASFGSSAETILTDSTYNLFEAVEVYSVQGQNLYLMIVECIGSNGRYFRSFTATSLGGTWTPQASTESNPFAGYANSGATWTKDISSGDLIRSSADQTKPVDPCNLQLLYQGRSPSSDNDSYNDLPYLPGLLTLAN

SEQ ID NO: 32 - protein, 275 aa, Bacillus amyloliquefaciens [sequence omitted]

SEQ ID NO: 33 - protein, 275 aa, Rasamsonia emersonii [sequence omitted]

SEQ ID NO: 34 - protein, 505 aa, Aspergillus niger
MRLSTSSLFLSVSLLGKLALGLSAAEWRTQSIYFLLTDRFGRTDNSTTATCDTGDQIYCGGSWQGIINHLDYIQGMGFTAIWISPITEQLPQDTADGEAYHGYWQQKIYDVNSNFGTADDLKSLSDALHARGMYLMVDVVPNHMGYAGNGNDVDYSVFDPFDSSSYFHPYCLITDWDNLTMVQDCWEGDTIVSLPDLNTTETAVRTIWYDWVADLVSNYSVDGLRIDSVLEVEPDFFPGYQEAAGVYCVGEVDNGNPALDCPYQKVLDGVLNYPIYWQLLYAFESSSGSISNLYNMIKSVASDCSDPTLLGNFIENHDNPRFASYTSDYSQAKNVLSYIFLSDGIPIVYAGEEQHYSGGKVPYNREATWLSGYDTSAELYTWIATTNAIRKLAISADSAYITYANDAFYTDSNTIAMRKGTSGSQVITVLSNKGSSGSSYTLTLSGSGYTSGTKLIEAYTCTSVTVDSSGDIPVPMASGLPRVLLPASVVDSSSLCGGSGRLYVE

SEQ ID NO: 35 - protein, 223 aa, Trichoderma reesei; identical to SEQ ID NO: 3

SEQ ID NO: 36 - protein, 483 aa, Bacillus licheniformis [sequence omitted]

SEQ ID NO: 37 - protein, 275 aa, Bacillus amyloliquefaciens [sequence omitted]

SEQ ID NO: 38 - protein, 223 aa, Trichoderma reesei; identical to SEQ ID NO: 3

SEQ ID NO: 39 - protein, 209 aa, Streptomyces fradiae
DKAGAVEIKRTPGTFNKFIAGGEAIYAAGGGRCSLGFNVRSSSGATYALTAGHCTEIASTWYTNSGQTSLLGTRAGTSFPGNDYGLIRHSNASAADGRVYLYNGSYRDITGAGNAYVGQTVQRSGSTTGLHSGRVTGLNATVNYGGGDIVSGLIQTNVCAEPGDSGGALFAGSTALGLTSGGSGNCRTGGTTFFQPVTEALSAYGVSII

SEQ ID NO: 40 - protein, 274 aa, Bacillus licheniformis
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 41 - protein, 413 aa, Buttiauxella sp.
NDTPASGYQVEKVVILSRHGVRAPTKMTQTMRDVTPNTWPEWPVKLGYITPRGEHLISLMGGFYRQKFQQQGILSQGSCPTPNSIYVWTDVAQRTLKTGEAFLAGLAPQCGLTIHHQQNLEKADPLFHPVKAGICSMDKTQVQQAVEKEAQTPIDNLNQHYIPSLALMNTTLNFSKSPWCQKHSADKSCDLGLSMPSKLSIKDNGNEVSLDGAIGLSSTLAEIFLLEYAQGMPQAAWGNIHSEQEWALLLKLHNVYFDLMERTPYIARHKGTPLLQAISNALNPNATESKLPDISPDNKILFIAGHDTNIANIAGMLNMRWTLPGQPDNTPPGGALVFERLADKSGKQYVSVSMVYQTLEQLRSQTPLSLNQPAGSVQLKIPGCNDQTAEGYCPLSTFTRVVSQSVEPGCQLQ

SEQ ID NO: 42 - protein, 413 aa, Buttiauxella sp.
SEAAPSGYHLDKAVILSRHGVRAPTKMTQLMRDVTPYTWPEWPVPLGYITPRGEHLVSLMGGFYRQYFQQQGLLSRDRCPTANDVYVWTDVNQRTRKTGEAFLAGLAPECDLTIHHQNDIKQVDPLFHPLKAGICSMDKTQVQQAVEKQAGMPIDKLNQHYRPELALMSNVLNFPKSPYCRQHSVEQPCDFANAFPSYLNISDDGNEVQLEGAVGLSSTLAEIFLLEYAQGMPVVAWGNIHSEQEWNDLLKLHNAQFDLMERTPYIAKHQGTPLLQTIVNALNSNTTESKLPDISPSVKILFLAGHDTNIANIGGMLGMTWTLPGQPDNTPPGGALLFELWSDKDGTQYVSVKMVYQTLAQLRNMTPLTLKEPAGSVPLKIPGCDDQTAEGYCPLDTFTRLVNQVVEPACQLP

SEQ ID NO: 43 - protein, 414 aa, Hafnia sp.
SETEPSGYQLEKVVILSRHGVRAPTKMTQTMRDVTPHQWPEWPVKLGYITPRGEHLISLMGGFYRERFQQQGLLPNDTCPTPDAVYVWTDVNQRTRKTGEAFLAGLAPQCDLAIHHQQNITQADPLFHPVKAGICSMNKSQTYAAVEKQAGGPIETLNQRYQAELALMSSVLDFPKSPYCQQHNIGKLCDFSQAMPSRLNISDDGNEVQLEGAVGLSSTLAEIFLLEYAQGMPVVAWGNIHNESQWKSLLNLHNAHFNLMHRTPYIAKHQGTPLLQAISNALNPNATESKLPDISPDNKILFIAGHDTNIANIGGMLGMNWTLPGQPDNTPPGGGLVFELWQNPDNHQQYVAVKMIYQTMDQLRNSEKLDLKSNPAGIVPIEIEGCENIGTDKLCQLDTFQKRVAQVIEPACQI

SEQ ID NO: 44 - protein, 432 aa, Escherichia coli
MKAILIPFLSLLIPLTPQSAFAQSEPELKLESVVIVSRHGVRAPTKATQLMQDVTPDAWPTWPVKLGWLTPRGGELIAYLGHYQRQRLVADGLLAKKGCPQPGQVAIIADVDERTRKTGEAFAAGLAPDCAITVHTQADTSSPDPLFNPLKTGVCQLDNANVTDAILSRAGGSIADFTGHRQTAFRELERVLNFPQSNLCLNREKQDESCSLTQALPSELKVSADNVSLTGAVSLASMLTEIFLLQQAQGMPEPGWGRITDSHQWNTLLSLHNAQFYLLQRTPEVARSRATPLLDLIMAALTPHPPQKQAYGVTLPTSVLFIAGHDTNLANLGGALELNWTLPGQPDNTPPGGELVFERWRRLSDNSQWIQVSLVFQTLQQMRDKTPLSLNTPPGEVKLTLAGCEERNAQGMCSLAGFTQIVNEARIPACSL

SEQ ID NO: 45 - protein, 410 aa, Escherichia coli
QSEEELKLESVVIVSRHGVRAPTKATQLMQDVTPYAWPTWPVKLGWLTPRGGELIAYLGHYWRQRLVADGLLPKKGCPQPGQVAIIADVDERTRKTGEAFAAGLAPDCAITVHTQADTSSPDPLFNPLKTGVCQLDVANVTDAILSRAGGSIADFTSHYQTAFRELERVLNFPQSPLCKNREKQNESCSLTQALPSELKVSADNVSLTGAWSLASMLTEIFLLQQAEGMPEPGWGRITDSHQWNTLLSLHNAYFDLLQRTPEVARSRATPLLDLIMAALTPHPPQKSAYGVTLPTSVLFIAGHDTNLANLGGALELNWTLPGQPDNYPPGGELVFERWRRLSDNSQWIQVSLVFQTLQQMRDKTPLSLNTPPGEVKLTLAGCEERNAQGMCSLAGFTQIVNEARIPACSL

SEQ ID NO: 46 - protein, 432 aa, Escherichia coli
MKAILIPFLSLLIPLTPQSAFAQSEPELKLESVVIVSRHGVRAPTKFTQLMQDVTPDAWPTWPVKLGELTPRGGELIAYLGHYWRQRLVADGLLPKCGCPQSGQVAIIADVDERTRKTGEAFAAGLAPDCAITVHHQADTSSPDPLFNPLKTGVCQLDVANVRRAILERAGGSIADFTRHYQTAFRELERVLNFPQSNLCLKREKQDESCSLTQALPSELKVSADDVSLTGAWSLASMLTEIFLLQQAQGMPEPGWGRITDSHQWNTLLSLHNAVFDLLQRTPEVARSRATPLLDLIKTALTPHPPQKQAYGVTLPTSVLFIAGHDTNLANLGGALELNWTLPGQPDNYPPGGELVFERWRRLSDNSQWIQVSLVFQTLQQMRDKTPLSLNTPPGEVKLTLAGCEERNAQGMCSLAGFTQIVNEARIPACSL

SEQ ID NO: 47 - protein, 410 aa, synthetic construct
QSEPELKLESVVIVSRHGVRAPTKATQLMQDVTPDAWPTWPVKLGELTPRGGELIAYLGHYWRQRLVADELLPKCGCPQPGQVAIIADVDERTRKTGEAFAAGLAPDCAITVHHQADTSSPDPLFNPLKTGVCQLDVANVTRAILERAGGSIADFTGHYQPAFRELERVLNFPQSPLCLKREKQDEPCSLTQALPSELKVSADNVSLTGAWSLASMLTEIFLLQQAQGMPEPGWGRITDSHQWNTLLSLHNAQFDLLQRTPEVARSRATPLLDLIKTALTPHPPQKQAYGVTLPTSVLFIAGHDTNLANLGGALELNWTLPGQPDNTPPGGELVFERWRRLSDNSQWIQVSLVFQTLQQMRDKTPLSLNTPPGEVKLTLPGCEERNAQGMCSLAGFTQIVNEARIPACSL

SEQ ID NO: 48 - protein, 440 aa, Escherichia coli
MKAILIPFLSLLIPLTPQSAFAQSEPELKLESVVIVSRHGVRAPTKATQLMQDVTPDAWPTWPVKLGWLTPRGGELIAYLGHYQRQRLVADGLLAKKGCPQSGQVAIIADVDERTRKTGEAFAAGLAPDCAITVHTQADTSSPDPLFNPLKTGVCQLDNANVTDAILSRAGGSIADFTGHRQTAFRELERVLNFPQSNLCLKREKQDESCSLTQALPSELKVSADNVSLTGAVSLASMLTEIFLLQQAQGMPEPGWGRITDSHQWNTLLSLHNAQFYLLQRTPEVARSRATPLLDLIMAALTPHPPQKQAYGVTLPTSVLFIAGHDTNLANLGGALELNWTLPGQPDNTPPGGELVFERWRRLSDNSQWIQVSLVFQTLQQMRDKTPLSLNTPPGEVKLTLAGCEERNAQGMCSLAGFTQIVNEARIPACSLRSHHHHHH

SEQ ID NO: 49 - protein, 223 aa, Trichoderma reesei; identical to SEQ ID NO: 3

SEQ ID NO: 50 - protein, 483 aa, Bacillus licheniformis [sequence omitted]

SEQ ID NO: 51 - protein, 275 aa, Bacillus amyloliquefaciens
AQSVPYGVSQIKAPALHSQGYTGSNVKVAVIDSGIDSSHPDLKVAGGASMVPSETNPFQDNNSHGTHVAGTVAALNNSIGVLGVAPSASLYAVKVLGADGSGQYSWIINGIEWAIANNMDVINMSLGGPSGSAALKAAVDKAVASGVVVVAAAGNEGTSGSSSTVGYPGKYPSVIAVGAVDSSNQRASFSSVGPELDVMAPGVSIQSTLPGNKYGALNGTSMASPHVAGAAALILSKHPNWTNTQVRSSLENTTTKLGDSFYYGKGLINVQAAAQ

SEQ ID NO: 52 - protein, 413 aa, Buttiauxella sp.; identical to SEQ ID NO: 42

SEQ ID NO: 53 - protein, 414 aa, Hafnia sp.; identical to SEQ ID NO: 43

SEQ ID NO: 54 - protein, 386 aa, Rasamsonia emersonii
AGLNTAAKAIGLKYFGTATDNPELSDTAYETQLNNTQDFGQLTPANSMKWDATEPEQNVFTFSAGDQIANLAKANGQMLRCHNLVWYNQLPSWVTSGSWTNETLLAAMKNHITNVVTHYKGQCYAWDVVNEALNDDGTYRSNVFYQYIGEAYIPIAFATAAAADPNAKLYYNDYNIEYPGAKATAAQNLVKLVQSYGARIDGVGLQSHFIVGETPSTSSQQQNMAAFTALGVEVAITELDIRMQLPETEALLTQQATDYQSTVQACANTKGCVGITVWDWTDKYSWVPSTFSGYGDACPWDANYQKKPAYEGILTGLGQTVTSTTYIISPTTSVGTGTTTSSGGSGGTTGVAQHWEQCGGLGWTGPTVCASGYTCTVINEYYSQCL

SEQ ID NO: 55 - protein, 410 aa, Escherichia coli; identical to SEQ ID NO: 45

SEQ ID NO: 56 - protein, 223 aa, Trichoderma reesei; identical to SEQ ID NO: 3

SEQ ID NO: 57 - protein, 411 aa, Escherichia coli
AQSEPELKLESVVIVSRHGVRAPTKFTQLMQDVTPDAWPTWPVKLGELTPRGGELIAYLGHYWRQRLVADGLLPKEGCPQSGQVAIIADVDERTRKTGEAFAAGLAPDCAITVHHQADTSSPDPLFNPLKTGVCQLDVANVRRAILERAGGSIADFTRHYQTAFRELERVLNFPQSNLCLKREKQDESCSLTQALPSELKVSADDVSLTGAWSLASMLTEIFLLQQAQGMPEPGWGRITDSHQWNTLLSLHNAVFDLLQRTPEVARSRATPLLDLIKTALTPHPPQKQAYGVTLPTSVLFIAGHDTNLANLGGALELNWTLPGQPDNYPPGGELVFERWRRLSDNSQWIQVSLVFQTLQQMRDKTPLSLNTPPGEVKLTLAGCEERNAQGMCSLAGFTQIVNEARIPACSL

SEQ ID NO: 58 - protein, 220 aa, Nonomuraea flexuosa
DTTITQNQTGYDNGYFYSFWTDAPGTVSMTLHSGGSYSTSWRNTGNFVAGKGWSTGGRRTVTYNASFNPSGNAYLTLYGWTRNPLVEYYIVESWGTYRPTGTYKGTVTTDGGTYDIYETWRYNAPSIEGTRTFQQFWSVRQQKRTSGTITIGNHFDAWARAGMNLGSHDYQIMATEGYQSSGSSTVSISEGGNPGNPGNPGNPGNPGNPGNPGGGCVATL

SEQ ID NO: 59 - protein, 432 aa, Escherichia coli; identical to SEQ ID NO: 44

SEQ ID NO: 60 - protein, 225 aa, Neocallimastix patriciarum
QSFCSSASHSGQSVKVTGNKVGTIGGWGYELWADSGNNSATFYSDGSFSCTFQNAGDYLCRSGLSFDSTKTPSQIGRMKADFKLVKQNSSNVGYSYVGVYGWTRSPLVEYYIVDNWLSPFPPGDWVGNKKFGSFTIDGAQYTVYENTRTGPSIDGDTTFNQYFSIRQQARDCGTIDISAHFDQWEKLGMTMGKLHEAKVLGEAGNVNGGASGTADFPYAKVYIGD

SEQ ID NO: 61 - protein, 521 aa, Bacillus amyloliquefaciens
MGLGKKLSVAVAASFMSLTISLPGVQAAENPQLKENLTNFVPKHSLVQSELPSVSDKAIKQYLKQNGKVFKGNPSERLKLIDQTTDDLGYKHFRYVPVVNGVPVKDSQVIIHVDKSNNVYAINGELNNDVSAKTANSKKLSANQALDHAYKAIGKSPEAVSNGTVANKNKAELKAAATKDGKYRLAYDVTIRYIEPEPANWEVTVDAETGKILKKQNKVEHAATTGTGTTLKGKTVSLNISSESGKYVLRDLSKPTGTQIITYDLQNREYNLPGTLVSSTTNQFTTSSQRAAVDAHYNLGKVYDYFYQKFNRNSYDNKGGKIVSSVHYGSRYNNAAWIGDQMIYGDGDGSFFSPLSGSMDVTAHEMTHGVTQETANLNYENQPGALNESFSDVFGYFNDTEDWDIGEDITVSQPALRSLSNPTKYGQPDNFKNYKNLPNTDAGDYGGVHTNSGIPNKAAYNTITKIGVNKAEQIYYRALTVYLTPSSTFKDAKAALIQSARDLYGSQDAASVEAAWNAVGL

SEQ ID NO: 62 - protein, 359 aa, Rhizomucor miehei
ASSFVQTSGPQFTLDGKPFYFEGTNAYYLMTSDQSNVKQVFSDMKSLGLPVVRTWLFNLGSDSVWFQQWDSSSNKMVINDNSDTGLGRIDYIIQQAASQDIKLIFTLNNNWEDYGGMDYYVKNFGGTYHDDFYTNTEMIDSFKEYISHVLNRENSLTGVKYKDDPTIFGWEIANEPRCVGSGDFPASSNCSTTVTTAWIKEISEYIKSIDSNHLVAVGDEGFFNRKGESDYEYNGGSGMDFDAILALSSIDFGTFHLYPEAWSKGTDSSWSVQWIKDHAAAQADADKPVIMEEYGLSTDALRVAQYPVWQGTVEDEDLAADAFWQIAVPCSTMDGFGICASDNDIATTVTNHADAMAKK